(19) United States
(12) Patent Application Publication
(10) Pub. No.: US 2010/0273804 A1
(43) Pub. Date: Oct. 28, 2010

(54) PYRIDAZINE DERIVATIVES USEFUL AS FUNGICIDES AND FOR THE TREATMENT OF CANCER

(75) Inventors: Clemens Lamberth, Stein (CH); Stephan Trah, Stein (CH); Raphael Dumeunier, Stein (CH); Sebastian Volker Wendeborn, Stein (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/809,604

(22) PCT Filed: Dec. 19, 2008
(86) PCT No.: PCT/EP08/10928

§ 371 Date: Jun. 21, 2010

(30) Foreign Application Priority Data
Dec. 21, 2007 (GB) 0725059.0

(51) Int. Cl. A61K 31/501; A01N 43/58; C07D 403/04; C07D 237/08; A01P 3/00; A61K 31/50; A61P 35/00

(52) U.S. Cl. 514/252.02; 514/247; 514/252.03; 544/224; 544/238; 544/239

(57) ABSTRACT

The present invention relates to novel pyridazine derivatives of formula (I) as active ingredients which have microbiocidal activity, in particular fungicidal activity: formula (I) wherein R¹ is C₁-C₆alkyl, C₁-C₆haloalkyl or C₃-C₆cycloalkyl; R² is hydrogen or an optionally substituted alkyl, aryl or heteroaryl; R³ is hydrogen, C₁-C₆alkyl or C₁-C₆haloalkyl; R⁴ is hydrogen, C₁-C₆alkyl or C₁-C₆haloalkyl; or R³ and R⁴ together can be part of a carbocyclic or heterocyclic 3- to 8-membered ring; R⁵ is optionally substituted aryl or heteroaryl; and R⁶ is hydroxy, halogen, C₁-C₆alkoxy, C₁-C₆haloalkoxy, C₁-C₆alkylthio or C₁-C₆haloalkylthio; or an agrochemically usable salt form thereof.

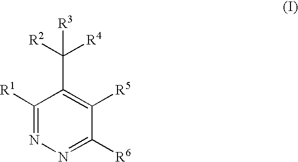

The present invention relates to novel pyridazine derivatives as active ingredients which have microbiocidal activity, in particular fungicidal activity. The invention also relates to preparation of these active ingredients, to novel heterocyclic derivatives used as intermediates in the preparation of these active ingredients, to preparation of these novel intermediates, to agrochemical compositions which comprise at least one of the novel active ingredients, to preparation of these compositions and to use of the active ingredients or compositions in agriculture or horticulture for controlling or preventing infestation of plants, harvested food crops, seeds or non-living materials by phytopathogenic microorganisms, preferably fungi.

In addition, the present invention also relates to the use of these novel pyridazine derivatives as plant growth regulators (PGRs).

Furthermore, the present invention also relates to compositions comprising the novel pyridazine derivatives that improve plants, a process which is commonly and hereinafter referred to as “plant health”.

The present invention further relates to the use of these novel pyridazine derivatives in the treatment of cancer and to fungicidal or pharmaceutical compositions comprising at least one of these compounds as active component.

These objects are achieved by the following compound of formula I:

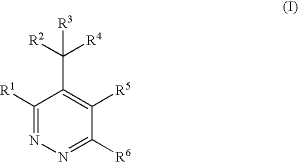

wherein
R¹ is C₁-C₆alkyl, C₁-C₆haloalkyl or C₃-C₆cycloalkyl;
R² is hydrogen or an optionally substituted alkyl, aryl or heteroaryl;
R³ is hydrogen, C₁-C₆alkyl or C₁-C₆haloalkyl;
R⁴ is hydrogen, C₁-C₆alkyl or C₁-C₆haloalkyl; or
R³ and R⁴ together can be part of a carbocyclic or heterocyclic 3- to 8-membered ring;
R⁵ is optionally substituted aryl or heteroaryl; and
R⁶ is hydroxy, halogen, C₁-C₆alkoxy, C₁-C₆haloalkoxy, C₁-C₆alkylthio or C₁-C₆haloalkylthio; or an agrochemically usable salt form thereof.

In the above definition aryl includes aromatic hydrocarbon rings like phenyl, naphthyl, anthracenyl, phenanthrenyl and biphenyl, with phenyl being preferred.

Heteroaryl stands for aromatic ring systems comprising mono-, bi- or tricyclic systems wherein at least one oxygen, nitrogen or sulfur atom is present as a ring member. Examples are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridinyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, quinolinyl, isoquinoinyl, phthalazinyl, quinoxalinyl, quinazolinyl, cinnolinyl and naphthyridinyl, with pyridinyl being preferred.

The above or below mentioned carbocyclic ring, heterocyclic ring, alkyl group, aryl group and heteroaryl group may be optionally substituted. This means that they may carry one or more identical or different substituents. Normally not more than three substituents are present at the same time. Examples of substituents are: halogen, alkyl, haloalkyl, cycloalkyl, cycloalkylalkyl, alkenyl, haloalkenyl, cycloalkenyl, alkynyl, haloalkynyl, alkyloxy, haloalkyloxy, cycloalkoxy, alkenyloxy, haloalkenyloxy, alkynyloxy, haloalkenyloxy, alkylthio, haloalkylthio, cycloalkylthio, alkenylthio, alkynylthio, alkylcarbonyl, haloalkylcarbonyl, cycloalkylcarbonyl, alkenylcarbonyl, alkynylcarbonyl, alkoxyalkyl, cyano, nitro, hydroxy, mercapto, amino, alkylamino, dialkylamino. Typical examples for optionally substituted aryl include 2-fluoro-phenyl, 3-fluoro-phenyl, 4-fluoro-phenyl, 2-chloro-phenyl, 3-chloro-phenyl, 4-chloro-phenyl, o-tolyl, m-tolyl, p-tolyl, 2-methoxy-phenyl, 3-methoxy-phenyl, 4-methoxy-phenyl, 2-trifluoromethyl-phenyl, 2,4-difluoro-phenyl, 2,6-difluoro-phenyl, 2,4-dichloro-phenyl, 2,6-dichloro-phenyl, 4-chloro-2-fluoro-phenyl, 2-chloro-4-fluoro-phenyl, 2-chloro-6-fluoro-phenyl, 2-fluoro-4-methoxy-phenyl, 2-fluoro-6-methoxy-phenyl, 4-fluoro-2-methoxy-phenyl, 2-fluoro-4-trifluoromethyl-phenyl, 2-fluoro-6-trifluoromethyl-phenyl, 4-fluoro-2-trifluoromethyl-phenyl, 2-chloro-4-methoxy-phenyl, 2-chloro-6-methoxy-phenyl, 4-chloro-2-methoxy-phenyl, 2-chloro-4-trifluoromethyl-phenyl, 2-chloro-6-trifluoromethyl-phenyl, 4-chloro-2-trifluoromethyl-phenyl, 2,3,4-trifluoro-phenyl, 2,3,6-trifluoro-phenyl, 2,4,5-trifluoro-phenyl, 2,4,6-trifluoro-phenyl, 2,6-difluoro-4-methoxy-phenyl, 2,4-difluoro-6-methoxy-phenyl, pentafluoro-phenyl. Typical examples for optionally substituted heteroaryl include 3-fluoro-pyridin-2-yl, 6-fluoro-pyridin-3-yl, 3-chloro-pyridin-2-yl, 6-chloro-pyridin-3-yl, 6-methyl-pyridin-3-yl, 3-methoxy-pyridin-2-yl, 6-methoxy-pyridin-3-yl, 3-trifluoromethyl-pyridin-2-yl, 3,5-difluoro-pyridin-2-yl, 3,5-dichloro-pyridin-2-yl, 3-chloro-5-fluoro-pyridin-2-yl, 5-chloro-3-fluoro-pyridin-2-yl, 3-fluoro-5-methoxy-pyridin-2-yl, 5-fluoro-3-methoxy-pyridin-2-yl, 3-fluoro-5-trifluoromethyl-pyridin-2-yl, 5-fluoro-3-trifluoromethyl-pyridin-2-yl, 3-chloro-5-methoxy-pyridin-2-yl, 5-chloro-3-methoxy-pyridin-2-yl, 3-chloro-5-trifluoromethyl-pyridin-2-yl, 5-chloro-3-trifluoromethyl-pyridin-2-yl or 5-chloro-pyrimidin-4-yl.

In the above definition halogen is fluorine, chlorine, bromine or iodine.

The alkyl, alkenyl or alkynyl radicals may be straight-chained or branched.

Alkyl on its own or as part of another substituent is, depending upon the number of carbon atoms mentioned, for example, methyl, ethyl, propyl, butyl, pentyl, hexyl and the isomers thereof, for example, isopropyl, isobutyl, sec-butyl, tert-butyl, isopentyl or tert-pentyl.

A haloalkyl group may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Cycloalkyl on its own or as part of another substituent is, depending upon the number of carbon atoms mentioned, for example, cyclopropyl, cyclobutyl, cyclopentyl or cyclohexyl.

Alkenyl on its own or as part of another substituent is, depending upon the number of carbon atoms mentioned, for example, ethenyl, allyl, 1-propenyl, buten-2-yl, buten-3-yl, penten-1-yl, penten-3-yl, hexen-1-yl or 4-methyl-3-pentenyl.

Alkynyl on its own or as part of another substituent is, depending upon the number of carbon atoms mentioned, for example, ethynyl, propyn-1-yl, propyn-2-yl, butyn-1-yl, butyn-2-yl, 1-methyl-2-butynyl, hexyn-1-yl or 1-ethyl-2-butynyl.

The presence of one or more possible asymmetric carbon atoms in a compound of formula I means that the compounds may occur in optically isomeric, that means enantiomeric or diastereomeric forms. As a result of the presence of a possible aliphatic C═C double bond, geometric isomerism, that means cis-trans or (E)-(Z) isomerism may also occur. Also atropisomers may occur as a result of restricted rotation about a single bond. Formula I is intended to include all those possible isomeric forms and mixtures thereof. The present invention intends to include all those possible isomeric forms and mixtures thereof for a compound of formula I.

In each case, the compounds of formula I according to the invention are in free form or in an agronomically usable salt form.

In a first embodiment, compounds of formula I according to the invention have R¹ which is C₁-C₅alkyl, C₁-C₅haloalkyl or C₃-C₅cycloalkyl.

In a second embodiment, compounds of formula I according to the invention have R² which is hydrogen or an optionally substituted C₁-C₆alkyl, phenyl, naphthyl, furyl, thienyl, pyridinyl or quinolinyl.

In a third embodiment, compounds of formula I according to the invention have R³ which is hydrogen, C₁-C₅alkyl or C₁-C₅haloalkyl.

In a fourth embodiment, compounds of formula I according to the invention have R⁴ which is hydrogen, C₁-C₅alkyl or C₁-C₅haloalkyl.

In a fifth embodiment, compounds of formula I according to the invention have R³ and R⁴ together which can be part of a carbocyclic or heterocyclic 3- to 7-membered ring.

In a sixth embodiment, compounds of formula I according to the invention have R⁵ which is an optionally substituted phenyl, pyridinyl, pyrimidinyl, thienyl or thiazolyl.

In a seventh embodiment, compounds of formula I according to the invention have R⁶ which is hydroxy, halogen, C₁-C₄alkoxy, C₁-C₄haloalkoxy, C₁-C₄alkylthio or C₁-C₄haloalkylthio.

Preferred subgroups of compounds of formula I according to the invention are those

wherein
R¹ is C₁-C₄alkyl, C₁-C₄haloalkyl or C₃-C₄cycloalkyl;
R² is hydrogen or an optionally substituted C₁-C₄alkyl, phenyl, furyl, thienyl, pyridinyl or quinolinyl;
R³ is hydrogen or C₁-C₅alkyl;
R⁴ is hydrogen or C₁-C₅alkyl; or
R³ and R⁴ together can be part of a carbocyclic 3- to 7-membered ring;
R⁵ is optionally substituted phenyl, pyridinyl, pyrimidinyl or thienyl; and
R⁶ is hydroxy, halogen, C₁-C₄alkoxy, C₁-C₄haloalkoxy or C₁-C₄alkylthio.

More preferred subgroups of compounds of formula I according to the invention are those wherein

R¹ is C₁-C₄alkyl, C₁-C₃haloalkyl;
R² is hydrogen or an optionally substituted C₁-C₄alkyl, phenyl, furyl, thienyl or pyridinyl;
R³ is hydrogen or C₁-C₄alkyl;
R⁴ is hydrogen or C₁-C₄alkyl; or
R³ and R⁴ together can be part of a carbocyclic 3- to 6-membered ring;
R⁶ is 2-fluoro-phenyl, 2-chloro-phenyl, 2-methoxy-phenyl, 2-trifluoromethyl-phenyl, 2,4-difluoro-phenyl, 2,6-difluoro-phenyl, 2,4-dichloro-phenyl, 2,6-dichloro-phenyl, 4-chloro-2-fluoro-phenyl, 2-chloro-4-fluoro-phenyl, 2-chloro-6-fluoro-phenyl, 2-fluoro-4-methoxy-phenyl, 2-fluoro-6-methoxy-phenyl, 4-fluoro-2-methoxy-phenyl, 2-fluoro-4-trifluoromethyl-phenyl, 2-fluoro-6-trifluoromethyl-phenyl, 4-fluoro-2-trifluoromethyl-phenyl, 2-chloro-4-methoxy-phenyl, 2-chloro-6-methoxy-phenyl, 4-chloro-2-methoxy-phenyl, 2-chloro-4-trifluoromethyl-phenyl, 2-chloro-6-trifluoromethyl-phenyl, 4-chloro-2-trifluoromethyl-phenyl, 2,3,4-trifluoro-phenyl, 2,3,6-trifluoro-phenyl, 2,4,5-trifluoro-phenyl, 2,4,6-trifluoro-phenyl, 2,6-difluoro-4-methoxy-phenyl, 2,4-difluoro-6-methoxy-phenyl, pentafluoro-phenyl, 3-fluoro-pyridin-2-yl, 3-chloro-pyridin-2-yl, 3-methoxy-pyridin-2-yl, 3-trifluoromethyl-pyridin-2-yl, 3,5-difluoro-pyridin-2-yl, 3,5-dichloro-pyridin-2-yl, 3-chloro-5-fluoro-pyridin-2-yl, 5-chloro-3-fluoro-pyridin-2-yl, 3-fluoro-5-methoxy-pyridin-2-yl, 5-fluoro-3-methoxy-pyridin-2-yl, 3-fluoro-5-trifluoromethyl-pyridin-2-yl, 5-fluoro-3-trifluoromethyl-pyridin-2-yl, 3-chloro-5-methoxy-pyridin-2-yl, 5-chloro-3-methoxy-pyridin-2-yl, 3-chloro-5-trifluoromethyl-pyridin-2-yl, 5-chloro-3-trifluoromethyl-pyridin-2-yl or 5-chloro-pyrimidin-4-yl; and
R⁶ is hydroxy, halogen, C₁-C₃alkoxy or C₁-C₃haloalkoxy.

Most preferred subgroups of compounds of formula I according to the invention are those wherein

R¹ is methyl, ethyl, isopropyl, tertiobutyl or trifluoromethyl;
R² is hydrogen or an optionally substituted C₁-C₄alkyl, phenyl, thienyl or pyridinyl;
R³ is hydrogen, methyl or ethyl;
R⁴ is hydrogen, methyl or ethyl; or
R³ and R⁴ together can be part of a carbocyclic 3- to 5-membered ring;
R⁵ is 2,4-difluoro-phenyl, 2,4-dichloro-phenyl, 2-chloro-6-fluoro-phenyl, 4-fluoro-2-methoxy-phenyl, 2-chloro-4-methoxy-phenyl, 2,4,5-trifluoro-phenyl, 2,4,6-trifluoro-phenyl, 2,6-difluoro-4-methoxy-phenyl, 2,4-difluoro-6-methoxy-phenyl, 3-trifluoromethyl-pyridin-2-yl, 3,5-dichloro-pyridin-2-yl or 5-chloro-pyrimidin-4-yl; and
R⁶ is hydroxy, chloro, fluoro, methoxy, ethoxy or trifluoromethoxy.

Especially preferred subgroups of compounds of formula I according to the invention are those wherein

R¹ is methyl;
R² is optionally substituted phenyl;
R³ is hydrogen;
R⁴ is hydrogen; or
R³ and R⁴ together can be part of a carbocyclic 3-membered ring;
R⁵ is 2,4,6-trifluoro-phenyl; and
R⁶ is hydroxy or chloro.

Preferred individual compounds are:
5-benzyl-6-methyl-4-(2,4,6-trifluoro-phenyl)-pyridazin-3-ol;
5-(4-fluoro-benzyl)-6-methyl-4-(2,4,6-trifluoro-phenyl)-pyridazin-3-ol;
5-(4-chloro-benzyl)-6-methyl-4-(2,4,6-trifluoro-phenyl)-pyridazin-3-ol;
3-chloro-5-(2-chloro-benzyl)-6-methyl-4-(2,4,6-trifluoro-phenyl)-pyridazine;
3-chloro-6-methyl-5-(2-methyl-benzyl)-4-(2,4,6-trifluoro-phenyl)-pyridazine;
3-chloro-5-(3-chloro-benzyl)-6-methyl-4-(2,4,6-trifluoro-phenyl)-pyridazine;
3-chloro-6-methyl-5-(3-methyl-benzyl)-4-(2,4,6-trifluoro-phenyl)-pyridazine;
3-chloro-5-(4-chloro-benzyl)-6-methyl-4-(2,4,6-trifluoro-phenyl)-pyridazine;
3-chloro-6-methyl-5-(4-methyl-benzyl)-4-(2,4,6-trifluoro-phenyl)-pyridazine;
3-chloro-5-(4-chloro-benzyl)-4-(2-chloro-6-fluoro-phenyl)-6-methyl-pyridazine;
3-chloro-4-(2-chloro-6-fluoro-phenyl)-6-methyl-5-(4-methyl-benzyl)-pyridazine;
3-chloro-5-(4-chloro-benzyl)-4-(2,6-difluoro-4-methoxy-phenyl)-6-methyl-pyridazine;
3-chloro-4-(2,6-difluoro-4-methoxy-phenyl)-6-methyl-5-(4-methyl-benzyl)-pyridazine;
3-chloro-5-(4-chloro-benzyl)-4-(3,5-dichloro-pyridin-2-yl)-6-methyl-pyridazine; and
3-chloro-4-(3,5-dichloro-pyridin-2-yl)-6-methyl-5-(4-methyl-benzyl)-pyridazine.

Certain pyridazine derivatives with aryl or heteroaryl groups in positions 4 and 5 have been proposed for controlling plant-destructive fungi, for example in WO 2005/121104, WO 2006/001175, WO 2007/066601 and WO 2007/080720. However, the action of those preparations is not satisfactory in all aspects of agricultural needs. Surprisingly, with the compounds of formula I, new kinds of fungicides having a high level of biological activity have now been found.

Compounds of formula (I.1), (I.2) and (I.3), in which R¹, R², R³, R⁴ and R⁵ have the meanings given above, are all examples of compounds of general formula (I) and can be made as shown in the following schemes.

The compounds of formula I.2, wherein R¹, R², R³, R⁴ and R⁵ are as defined for formula I, X is oxygen or sulfur and R⁷ is C₁-C₆alkyl or C₁-C₆haloalkyl, can be obtained by transformation of a compound of formula I.1, wherein R¹, R², R³, R⁴ and R⁵ are as defined for formula I and Hal is halogen, preferably chlorine or bromine, with an alcohol or a thiol R⁷XH, wherein R⁷ is C₁-C₆alkyl or C₁-C₆haloalkyl and X is oxygen or sulfur, and a base or with a sodium alkoxide or thioalkoxide NaXR⁷, wherein X is oxygen or sulfur and R⁷ is C₁-C₆alkyl or C₁-C₆haloalkyl.

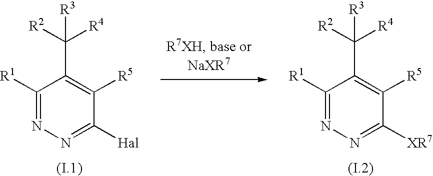

The compounds of formula I.1, wherein R¹, R², R³, R⁴ and R⁵ are as defined for formula I and Hal is halogen, preferably chlorine or bromine, can be obtained by transformation of a compound of formula I.3, wherein R¹, R², R³, R⁴ and R⁵ are as defined for formula I, with a phosphorus oxyhalide PO(Hal)₃, e.g. phosphorus oxychloride or phosphorus oxybromide, or a thionyl halide SO(Hal)₂, e.g. thionyl chloride or thionyl bromide.

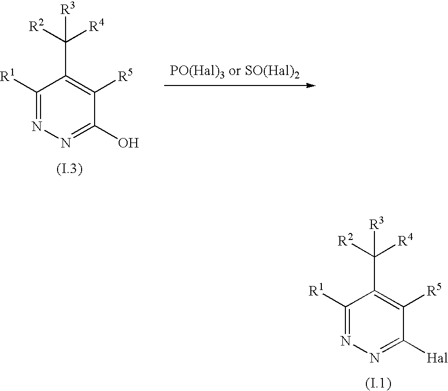

The compounds of formula I.3, wherein R¹, R², R³, R⁴ and R⁵ are as defined for formula I, can be obtained by transformation of a compound of formula II, wherein R¹, R², R³, R⁴ and R⁵ are as defined for formula I, with a hydrazine derivative, e.g. hydrazine hydrate.

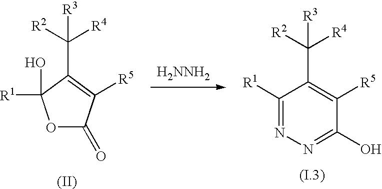

The compounds of formula II, wherein R¹, R², R³, R⁴ and R⁵ are as defined for formula I, can be obtained by transformation of a compound of formula III, wherein R¹, R², R³, R⁴ and R⁵ are as defined for formula I, by oxidation with oxygen, air or 3-chloroperbenzoic acid (mCPBA).

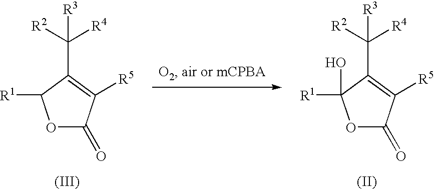

The compounds of formula III, wherein R¹, R², R³, R⁴ and R⁵ are as defined for formula I, can be obtained by transformation of a compound of formula IV, wherein R¹, R², R³, R⁴ and R⁵ are as defined for formula I, with a base, e.g. pyridine, triethylamine, diisopropylethylamine, 1,5-diazabicyclo[4.3.0]non-5-ene or 1,8-diazabicyclo[5.4.0]undec-7-ene.

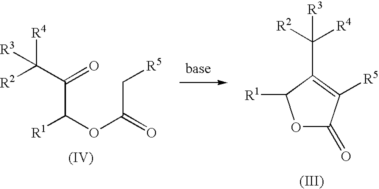

The compounds of formula IV, wherein R¹, R², R³, R⁴ and R⁵ are as defined for formula I, can be obtained by transformation of a compound of formula V, wherein R¹, R², R³ and R⁴ are as defined for formula I and Hal is halogen, preferably chlorine or bromine, with a compound of formula VI, wherein R⁵ is as defined for formula I, and a base, e.g. pyridine, triethylamine, diisopropylethylamine, 1,5-diazabicyclo[4.3.0]non-5-ene or 1,8-diazabicyclo[5.4.0]undec-7-ene.

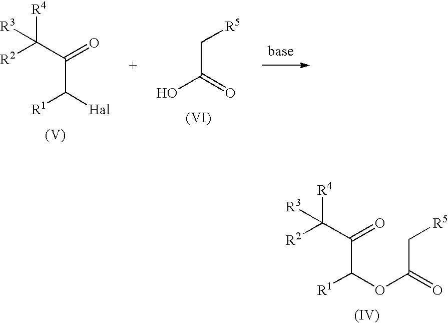

Surprisingly, it has now been found that the novel compounds of formula I have, for practical purposes, a very advantageous level of biological activity for protecting plants against diseases that are caused by fungi as well as by bacteria and viruses.

The compounds of formula I can be used in unmodified form or, preferably, together with carriers and adjuvants conventionally employed in the art of formulation.

Therefore the invention also relates to compositions for controlling and protecting against phytopathogenic micro-organisms, comprising a compound of formula I and an inert carrier, and to a method of controlling or preventing infestation of useful plants by phytopathogenic micro-organisms, wherein a composition, comprising a compound of formula I as active ingredient and an inert carrier, is applied to the plants, to parts thereof or the locus thereof.

In addition, the invention could be used to protect non-living materials from fungal attack, e.g. lumber, wall boards and paint.

To this end compounds of formula I and inert carriers are conveniently formulated in known manner to mollifiable concentrates, coat able pastes, directly spray able or dilatable solutions, dilute emulsions, wet table powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or pacifiers as well as fertilizers, micronutrient donors or other formulations for obtaining special effects.

Suitable carriers and adjuvants can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of formula I or compositions, comprising a compound of formula I as active ingredient and an inert carrier, can be applied to the locus of the plant or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides or mixtures of several of these preparations, if desired together with further carriers, surfactants or application promoting adjuvants customarily employed in the art of formulation.

A preferred method of applying a compound of formula I, or a composition, comprising a compound of formula I as active ingredient and an inert carrier, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, i.e. a composition comprising the compound of formula I and, if desired, a solid or liquid adjuvant, is prepared in a known manner, typically by intimately mixing and/or grinding the compound with extenders, for example solvents, solid carriers and, optionally, surface-active compounds (surfactants).

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds. The rate of application for the desired action can be determined by experiments. It depends for example on the type of action, the developmental stage of the useful plant, and on the application (location, timing, application method) and can, owing to these parameters, vary within wide limits.

The invention relates to a method of controlling or preventing infestation of useful plants by phytopathogenic micro-organisms, wherein a compound of formula I is applied as active ingredient to the plants, to parts thereof or the locus thereof. The compounds of formula I according to the invention are distinguished by excellent activity at low rates of application, by being well tolerated by plants and by being environmentally safe. They have very useful curative, preventive and systemic properties and are used for protecting numerous useful plants. The compounds of formula I can be used to inhibit or destroy the diseases that occur on plants or parts of plants (fruit, blossoms, leaves, stems, tubers, roots) of different crops of useful plants, while at the same time protecting also those parts of the plants that grow later e.g. from phytopathogenic micro-organisms.

It is also possible to use compounds of formula I as dressing agents for the treatment of plant propagation material, in particular of seeds (fruit, tubers, grains) and plant cuttings (e.g. rice), for the protection against fungal infections as well as against phytopathogenic fungi occurring in the soil.

Furthermore the compounds of formula I according to the invention may be used for controlling fungi in related areas, for example in the protection of technical materials, including wood and wood related technical products, in food storage or in hygiene management.

Within the scope of the invention, useful plants and/or target crops to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as ornamentals.

The term “useful plants” and/or “target crops” are to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” and/or “target crops” are to be understood as including also useful plants which have been so transformed by the use of recombinant DNA techniques that they are capable of synthesising one or more selectively acting toxins, such as are known, for example, from toxin-producing bacteria, especially those of the genus Bacillus.

The term “useful plants” and/or “target crops” are to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I are, for example, effective against the phytopathogenic fungi of the following classes: Fungi imperfecti (e.g. Alternaria spp.), Basidiomycetes (e.g. Corticium spp., Ceratobasidium spp., Waitea spp., Thanatephorus spp., Rhizoctonia spp., Hemileia spp., Puccinia spp., Phakopsora spp., Ustilago spp., Tilletia spp.), Ascomycetes (e.g. Venturia spp., Blumeria spp., Erysiphe spp., Podosphaera spp., Uncinula spp., Monilinia spp., Sclerotinia spp., Colletotrichum spp., Glomerella spp., Fusarium spp., Gibberella spp., Monographella spp., Phaeosphaeria spp., Mycosphaerella spp., Cercospora spp., Pyrenophora spp., Rhynchosporium spp., Magnaporthe spp., Gaeumannomyces spp., Oculimacula spp., Ramularia spp., Botryotinia spp.) and Oomycetes (e.g. Phytophthora spp., Pythium spp., Plasmopara spp., Peronospora spp., Pseudoperonospora spp. Bremia spp). Outstanding activity is observed against powdery mildews (e.g. Uncinula necator), rusts (e.g. Puccinia spp.) and leaf spots (e.g. Mycosphaerella spp.). Furthermore, the novel compounds of formula I are effective against phytopathogenic gram negative and gram positive bacteria (e.g. Xanthomonas spp, Pseudomonas spp, Erwinia amylovora, Ralstonia spp.) and viruses (e.g. tobacco mosaic virus).

The compounds of formula I are normally used in the form of fungicidal compositions for controlling or protecting against phytopathogenic microorganisms, comprising as active ingredient at least one compound of formula I or at least one preferred individual compound as above-defined, in free form or in agrochemically usable salt form, and at least one of the above-mentioned adjuvants.

Said fungicidal compositions for controlling or protecting against phytopathogenic microorganisms, comprising as active ingredient at least one compound of formula I or at least one preferred individual compound as above-defined, in free form or in agrochemically usable salt form, and at least one of the above-mentioned adjuvants can be mixed with other fungicides, resulting in some cases in unexpected synergistic activities. Mixing components which are particularly preferred are:

Azoles, such as azaconazole, BAY 14120, bitertanol, bromuconazole, cyproconazole, difenoconazole, diniconazole, epoxiconazole, fenbuconazole, fluquinconazole, flusilazole, flutriafol, hexaconazole, imazalil, imibenconazole, ipconazole, metconazole, myclobutanil, pefurazoate, penconazole, prothioconazole, pyrifenox, prochloraz, propiconazole, simeconazole, tebuconazole, tetraconazole, triadimefon, triadimenol, triflumizole, triticonazole;

Pyrimidinyl carbinoles, such as ancymidol, fenarimol, nuarimol;

2-amino-pyrimidines, such as bupirimate, dimethirimol, ethirimol;

Morpholines, such as dodemorph, fenpropidine, fenpropimorph, spiroxamine, tridemorph;

Anilinopyrimidines, such as cyprodinil, mepanipyrim, pyrimethanil;

Pyrroles, such as fenpiclonil, fludioxonil;

Phenylamides, such as benalaxyl, furalaxyl, metalaxyl, R-metalaxyl, ofurace, oxadixyl;

Benzimidazoles, such as benomyl, carbendazim, debacarb, fuberidazole, thiabendazole;

Dicarboximides, such as chlozolinate, dichlozoline, iprodione, myclozoline, procymidone, vinclozoline;

Carboxamides, such as boscalid, carboxin, fenfuram, flutolanil, mepronil, oxycarboxin, penthiopyrad, thifluzamide; guanidines, such as guazatine, dodine, iminoctadine;

Strobilurines, such as azoxystrobin, dimoxystrobin, enestroburin, fluoxastrobin, kresoxim-methyl, metominostrobin, trifloxystrobin, orysastrobin, picoxystrobin, pyraclostrobin;

Dithiocarbamates, such as ferbam, mancozeb, maneb, metiram, propineb, thiram, zineb, ziram;

N-halomethylthiotetrahydrophthalimides, such as captafol, captan, dichlofluanid, fluoromides, folpet, tolyfluanid;

Copper-compounds, such as Bordeaux mixture, copper hydroxide, copper oxychloride, copper sulfate, cuprous oxide, mancopper, oxine-copper;

Nitrophenol-derivatives, such as dinocap, nitrothal-isopropyl;

Organo-phosphorus-derivatives, such as edifenphos, iprobenphos, isoprothiolane, phosdiphen, pyrazophos, tolclofos-methyl;

Pyridazine-derivatives which are known and may be prepared by methods as described in WO 05/121104, WO 06/001175 and WO 07/066,601, such as 3-chloro-5-(4-chloro-phenyl)-6-methyl-4-(2,4,6-trifluoro-phenyl)-pyridazine (formula P.1), 3-chloro-6-methyl-5-p-tolyl-4-(2,4,6-trifluoro-phenyl)-pyridazine (formula P.2) and 3-chloro-4-(3-chloro-5-methoxy-pyridin-2-yl)-5-(4-chloro-phenyl)-6-methyl-pyridazine (formula P.3);

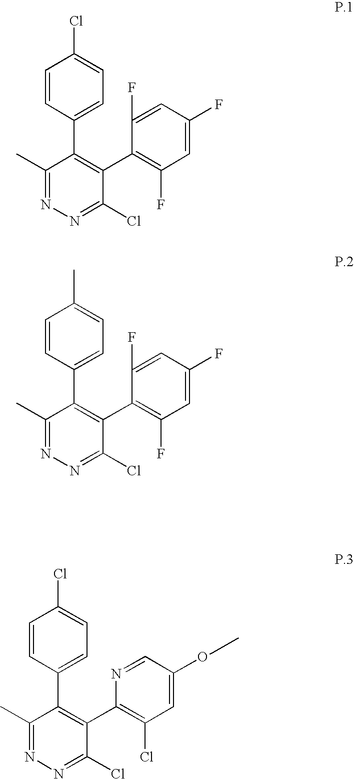

Triazolopyrimidine derivatives which are known and may be prepared by methods as described in WO98/46607, such as 5-chloro-7-(4-methyl-piperidin-1-yl)-6-(2,4,6-trifluoro-phenyl)-[1,2,4]triazolo[1,5-a]pyrimidine (formula T.1);

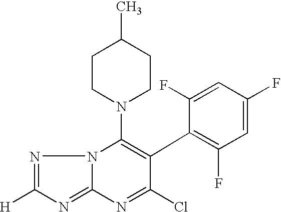

Carboxamide derivatives which are known and may be prepared by methods as described in WO04/035589, WO06/37632, WO03/074491 or WO03070705, such as 3-difluoromethyl-1-methyl-1H-pyrazole-4-carboxylic acid (9-isopropyp-1,2,3,4-tetrahaydro-1,4-methano-naphthalen-5-yl)-amide (formula U.1), 3-difluoromethyl-1-methyl-1H-pyrazole-4-carboxylic acid (2-bicyclopropyl-2-yl-phenyl)-amide (formula U.2) or N-(3′,4′-dichloro-5-fluoro-1,1′-biphenyl-2-yl)-3-(difluoromethyl)-1-methyl-1H-pyrazole-4-carboxamide;

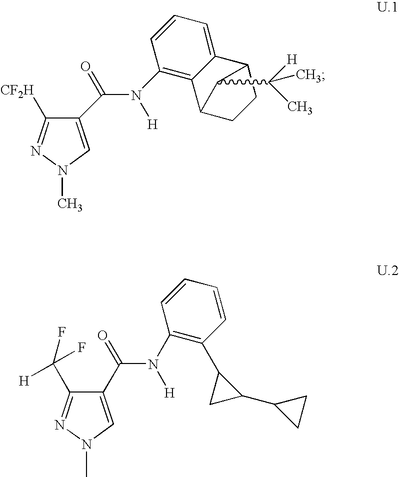

Benzamide derivatives which are known and may be prepared by methods as described in WO 2004/016088, such as N-{-2-[3-chloro-5-(trifluoromethyl)-2-pyridinyl]ethyl}-2-trifluoromethylbenzamide, which is also known under the name fluopyram (formula V.1);

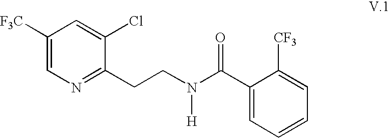

and various others, such as acibenzolar-S-methyl, anilazine, benthiavalicarb, blasticidin-S, chinomethionate, chloroneb, chlorothalonil, cyflufenamid, cymoxanil, dichlone, diclocymet, diclomezine, dicloran, diethofencarb, dimethomorph, flumorph, dithianon, ethaboxam, etridiazole, famoxadone, fenamidone, fenoxanil, fentin, ferimzone, fluazinam, fluopicolide, flusulfamide, fenhexamid, fosetyl-aluminium, hymexazol, iprovalicarb, cyazofamid, kasugamycin, mandipropamid, methasulfocarb, metrafenone, nicobifen, pencycuron, phthalide, polyoxins, probenazole, propamocarb, proquinazid, pyroquilon, quinoxyfen, quintozene, sulfur, tiadinil, triazoxide, tricyclazole, triforine, validamycin, zoxamide and glyphosate.

Another aspect of invention is related to the use of a compound of formula I or of a preferred individual compound as above-defined, of a composition comprising at least one compound of formula I or at least one preferred individual compound as above-defined, or of a fungicidal mixture comprising at least one compound of formula I or at least one preferred individual compound as above-defined, in admixture with other fungicides, as described above, for controlling or preventing infestation of plants, harvested food crops or non-living materials by phytopathogenic microorganisms, preferably fungal organisms.

A further aspect of invention is related to a method of controlling or preventing an infestation of crop plants, harvested food crops or of non-living materials by phytopathogenic or spoilage microorganisms or organisms potentially harmful to man, especially fungal organisms, which comprises the application of a compound of formula I or of a preferred individual compound as above-defined, as active ingredient to the plants, to parts of the plants or to the locus thereof, to seeds or to any part of the non-living materials.

Controlling or preventing means reducing the infestation of crop plants or of non-living materials by phytopathogenic or spoilage microorganisms or organisms potentially harmful to man, especially fungal organisms, to such a level that an improvement is demonstrated.

Surprisingly, the pyridazine compounds of formula I according to the invention, in particular the individual pyridazine compounds described in the above description as being preferred, also present a plant growth regulator (PGR) activity. Therefore, the present invention also relates to the use of these novel pyridazine derivatives as plant growth regulators (PGRs).

Plant growth regulators (PGRs) are generally any substances or mixtures of substances intended to accelerate or retard the rate of growth or maturation, or otherwise alter the development of plants or their produce.

Plant growth regulators (PGRs) affect growth and differentiation of plants.

More specifically, various plant growth regulators (PGRs) can, for example, reduce plant height, stimulate seed germination, induce flowering, darken leaf coloring, change the rate of plant growth and modify the timing and efficiency of fruiting.

Furthermore, the present invention also relates to compositions comprising the novel pyridazine derivatives of the present invention that improve plants, a process which is commonly and hereinafter referred to as “plant health”.

For example, advantageous properties that may be mentioned are improved crop characteristics including: emergence, crop yields, protein content, increased vigour, faster maturation, increased speed of seed emergence, improved nitrogen utilization efficiency, improved water use efficiency, improved oil content and/or quality, improved digestibility, faster ripening, improved flavor, improved starch content, more developed root system (improved root growth), improved stress tolerance (e.g. against drought, heat, salt, light, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Advantageous properties, obtained especially from treaded seeds, are e.g. improved germination and field establishment, better vigor, more homogeneous field establishment.

Advantageous properties, obtained especially from foliar and/or in-furrow application are e.g. improved plant growth and plant development, better growth, more tillers, greener leafes, largers leaves, more biomass, better roots, improved stress tolerance of the plants, more grain yield, more biomass harvested, improved quality of the harvest (content of fatty acids, metabolites, oil etc), more marketable products (e.g. improved size), improved process (e.g. longer shelf-life, better extraction of compounds), improved quality of seeds (for being seeded in the following seasons for seed production); or any other advantages familiar to a person skilled in the art.

It is therefore an object of the present invention to provide a method which solves the problems outlined above.

The present invention relates to plant-protecting active ingredients that are pyridazine compounds of formula I according to the invention, in particular the individual pyridazine compounds described in the above description as being preferred, and mixtures with increased efficacy and to a method of improving the health of plants by applying said compounds and mixtures to the plants or the locus thereof.

The action of the compounds of formula I goes beyond the known fungicidal action. The pyridazine compounds of formula I according to the invention, in particular the individual pyridazine compounds described in the above description as being preferred compounds exhibit plant health

The term plant health comprises various sorts of improvements of plants that are not connected to the control of harmful fungi.

In another aspect, the present invention relates to a composition comprising at least one compound of formula I or at least one preferred individual compound as above-defined and/or at least one pharmaceutically acceptable salt thereof, at least one pharmaceutically acceptable carrier and/or at least one pharmaceutically acceptable diluent.

In a further aspect, the present invention also relates to a compound of formula I or a preferred individual compound as above-defined, or a pharmaceutically acceptable salt thereof for use as a medicament.

In a preferred aspect, the present invention also relates to a compound of formula I or of a preferred individual compound as above-defined, or a pharmaceutically acceptable salt thereof for the treatment of cancer.

In an additional aspect, the present invention also relates to the use of a compound formula I or of a preferred individual compound as above-defined, or a pharmaceutically acceptable salt thereof in the manufacture of a medicament for the treatment of cancer.

In a particular aspect, the present invention also relates to a method of treating cancer in a subject in need thereof, comprising administering a compound formula I or a preferred individual compound as above-defined to said subject in an amount effective to treat said cancer.

The invention further provides fungicidal or pharmaceutical compositions comprising a compound of formula I or a preferred individual compound as above-defined, and/or their agriculturally or pharmaceutically acceptable salts and suitable carriers.

Suitable pharmaceutically acceptable carriers are described below.

The pyridazine compounds of formula I according to the invention, in particular the individual pyridazine compounds described in the above description as being preferred, and/or their pharmaceutically acceptable salts are suitable for the treatment, inhibiton or control of growth and/or propagation of tumor cells and the disorders associated therewith.

Accordingly, they are suitable for cancer therapy in warmblooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The pyridazine compounds of formula I according to the invention, in particular the individual pyridazine compounds described in the above description as being preferred, and/or their pharmaceutically acceptable salts are suitable for the therapy of cancer or cancerous disorders of the following organs: breast, lung, intestine, prostate, skin (melanoma), kidney, bladder, mouth, larynx, oesophagus, stomach, ovaries, pancreas, liver and brain.

In addition to pyridazine compounds of formula I according to the invention, in particular the individual pyridazine compounds described in the above description as being preferred, and/or its pharmaceutically acceptable salt, the pharmaceutical compositions according to the invention comprise at least optionally a suitable carrier.

“Pharmaceutically acceptable” means compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Suitable carriers are, for example, solvents, carriers, excipients, binders and the like customarily used for pharmaceutical formulations, which are described below in an exemplary manner for individual types of administration.

“Pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include:
sugars, such as lactose, glucose and sucrose;
starches, such as corn starch and potato starch;
cellulose, and its derivatives, such as sodium carboxymethyl cellulose, ethyl cellulose and cellulose acetate;
powdered tragacanth;
malt;
gelatin;
talc;
excipients, such as cocoa butter and suppository waxes;
oils, such as peanut oil, cottonseed oil, safflower oil, sesame oil, olive oil, corn oil and soybean oil;
glycols, such as propylene glycol;
polyols, such as glycerin, sorbitol, mannitol and polyethylene glycol;
esters, such as ethyl oleate and ethyl laurate;
agar; buffering agents, such as magnesium hydroxide and aluminum hydroxide;
alginic acid;
pyrogen-free water;
isotonic saline;
Ringer's solution;
ethyl alcohol;
phosphate buffer solutions; and
other non-toxic compatible substances employed in pharmaceutical formulations.

The pyridazine compounds of formula I according to the invention, in particular the individual pyridazine compounds described in the above description as being preferred (the active compound), can be administered in a customary manner, for example orally, intravenously, intramuscularly or subcutaneously.

For oral administration, the active compound can be mixed, for example, with an inert diluent or with an edible carrier; it can be embedded into a hard or soft gelatin capsule, it can be compressed to tablets or it can be mixed directly with the food/feed.

The active compound can be mixed with excipients and administered in the form of indigestible tablets, buccal tablets, pastilles, pills, capsules, suspensions, potions, syrups and the like.

Such preparations should contain at least 0.1% of active compound.

The composition of the preparation may, of course, vary.

It usually comprises from 2 to 60% by weight of active compound, based on the total weight of the preparation in question (dosage unit).

Preferred preparations of the pyridazine compounds of formula I according to the invention, in particular the individual pyridazine compounds described in the above description as being preferred, comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like.

Capsules may furthermore comprise a liquid carrier.

Other substances which modify the properties of the dosage unit may also be used.

For example, tablets, pills and capsules may be coated with schellack, sugar or mixtures thereof.

In addition to the active compound, syrups or potions may also comprise sugar (or other sweeteners), methyl- or propylparaben as preservative, a colorant and/or a flavor.

The components of the active compound preparations must, of course, be pharmaceutically pure and nontoxic at the quantities employed.

Furthermore, the active compounds can be formulated as preparations with a controlled release of active compound, for example as delayed-release preparations.

The active compounds can also be administered parenterally or intraperitoneally.

Solutions or suspensions of the active compounds or their salts can be prepared with water using suitable wetting agents, such as hydroxypropylcellulose.

Dispersions can also be prepared using glycerol, liquid polyethylene glycols and mixtures thereof in oils.

Frequently, these preparations furthermore comprise a preservative to prevent the growth of microorganisms.

Preparations intended for injections comprise sterile aqueous solutions and dispersions and also sterile powders for preparing sterile solutions and dispersions.

The preparation has to be sufficiently liquid for injection.

It has to be stable under the preparation and storage conditions and it has to be protected against contamination by microorganisms.

The carrier may be a solvent or a dispersion medium, for example, water, ethanol, a polyol (for example glycerol, propylene glycol or liquid polyethylene glycol), a mixture thereof and/or a vegetable oil.

Pharmaceutical compositions of this invention suitable for parenteral administration comprise an pyridazine compound of formula I according to the invention, in particular an individual pyridazine compounds described in the above description as being preferred, in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions of the present invention may be given by any suitable means of administration including orally, parenterally, topically, transdermally or rectally. They are of course given by forms suitable for each administration route. For example, they are administered in tablets or capsule form, by injection, inhalation, eye lotion, ointment, suppository, administration by injection, infusion or inhalation; topical by lotion or ointment; and rectal by suppositories. Topical or parenteral administration is preferred.

The following non-limiting examples illustrate the above-described invention in more detail.

### EXAMPLE 1

### This example illustrates the preparation of 3-chloro-5-(4-chloro-benzyl)-6-methyl-4-(2,4,6-trifluoro-phenyl)-pyridazine (Compound No. I.k.158)

### a) Preparation of 3-bromo-1-(4-chloro-phenyl)-butan-2-one

A suspension of copper(II) bromide (26.6 g) in 200 ml of a mixture of chloroform and ethyl acetate 1:1 is heated to reflux. At this temperature, a solution of 1-(4-chloro-phenyl)-butan-2-one (21.8 g) in 40 ml of a mixture of chloroform and ethyl acetate 1:1 is added dropwise. After heating the reaction mixture for further 2 h to reflux, it is cooled to room temperature and filtered. The residue is washed with ethyl acetate and the combined filtrate is evaporated. The remainder is taken up in carbon tetrachloride and filtered again. The filtrate is evaporated and the remainder is purified by chromatography on silica gel, using a mixture of heptane/ethyl acetate 9:1 as eluent to deliver 3-bromo-1-(4-chloro-phenyl)-butan-2-one as a brown oil.

### b) Preparation of 4-(4-chloro-benzyl)-5-hydroxy-5-methyl-3-(2,4,6-trifluoro-phenyl)-5H-furan-2-one (Compound No. II.k.53)

A mixture of 3-bromo-1-(4-chloro-phenyl)-butan-2-one (12 g), 2,4,6-trifluoro-phenylacetic acid (9.6 g), 7.0 ml of triethylamine and 120 ml of acetonitrile is stirred for 16 h at room temperature. Subsequently 16 ml of 1,8-diazabicyclo[5.4.0]undec-7-ene (DBU) are added under cooling and stirring is continued for further 2 h. Then air is blown through the reaction mixture for 6 h. An aqueous ammonium chloride solution is added and the mixture is extracted with ethyl acetate. The combined organic layer is washed with a saturated aqueous sodium bicarbonate solution and with brine, dried over sodium sulfate and evaporated under reduced pressure. The remainder is purified by chromatography on silica gel, using a mixture of heptane/ethyl acetate 4:1 as eluent to obtain 4-(4-chloro-benzyl)-5-hydroxy-5-methyl-3-(2,4,6-trifluoro-phenyl)-5H-furan-2-one (Compound No. II.k.53) as light yellow crystals, m.p. 127-129° C.

### c) Preparation of 5-(4-chloro-benzyl)-6-methyl-4-(2,4,6-trifluoro-phenyl)-2H-pyridazin-3-one (Compound No. I.k.157)

A mixture of 4-(4-chloro-benzyl)-5-hydroxy-5-methyl-3-(2,4,6-trifluoro-phenyl)-5H-furan-2-one (Compound No. II.k.53, 6.3 g), 0.9 ml of hydrazine hydrate and 35 ml of 1-butanol is heated for 8 h to 120° C. Subsequently, the mixture is cooled to 0° C. The hereby obtained solid is filtered and washed with hexane to obtain 5-(4-chloro-benzyl)-6-methyl-4-(2,4,6-trifluoro-phenyl)-2H-pyridazin-3-one (Compound No. I.k.157) as light yellow crystals, m.p. 212-215° C.

### d) A mixture of 5-(4-chloro-benzyl)-6-methyl-4-(2,4,6-trifluoro-phenyl)-2H-pyridazin-3-one

(Compound No. I.k.157, 2.5 g) and 10 ml of phosphorus oxychloride are mixed and heated at 110° C. for 1 h. After cooling the reaction mixture is evaporated under reduced pressure. The remainder is taken up with ethyl acetate and water and the phases are separated. The organic layer is washed with water and brine, dried over sodium sulfate and evaporated under reduced pressure. The residue is purified by chromatography on silica gel, using a mixture of heptane/ethyl acetate 3:1 as eluent to deliver 3-chloro-5-(4-chloro-benzyl)-6-methyl-4-(2,4,6-trifluoro-phenyl)-pyridazine (Compound No. I.k.158) as a light yellow oil.

### EXAMPLE 2

### This example illustrates the preparation of 4-(4-chloro-benzyl)-6-methoxy-3-methyl-5-(2,4,6-trifluoro-phenyl)-pyridazine (Compound No. I.k.159)

A mixture of 3-chloro-5-(4-chloro-benzyl)-6-methyl-4-(2,4,6-trifluoro-phenyl)-pyridazine (Compound No. I.k.158, 0.8 g), sodium methoxide (30% solution in methanol, 0.5 g) and 10 ml of methanol is heated for 16 h to 60° C. Subsequently the reaction mixture is cooled, diluted with water and extracted with ethyl acetate. The combined organic layer is washed with water and brine, dried over sodium sulfate and evaporated under reduced pressure. The remainder is purified by chromatography on silica gel, using a mixture of heptane/ethyl acetate 9:1 as eluent to obtain 4-(4-chloro-benzyl)-6-methoxy-3-methyl-5-(2,4,6-trifluoro-phenyl)-pyridazine (Compound No. I.k.159).

Tables 1 and 2 below illustrate examples of individual compounds of formula I and formula II according to the invention.

TABLE 1
individual compounds of formula I according to the invention

| Com- | | | |
|---|---|---|---|
| pound | | | |
| No. | R¹ | R⁵ | R⁶ |
| 001 | CH₃ | 2-fluoro-phenyl | OH |
| 002 | CH₃ | 2-fluoro-phenyl | Cl |
| 003 | CH₃ | 2-fluoro-phenyl | OCH₃ |
| 004 | CH₂CH₃ | 2-fluoro-phenyl | OH |
| 005 | CH₂CH₃ | 2-fluoro-phenyl | Cl |
| 006 | CH₂CH₃ | 2-fluoro-phenyl | OCH₃ |
| 007 | CH₃ | 2-chloro-phenyl | OH |
| 008 | CH₃ | 2-chloro-phenyl | Cl |
| 009 | CH₃ | 2-chloro-phenyl | OCH₃ |
| 010 | CH₂CH₃ | 2-chloro-phenyl | OH |
| 011 | CH₂CH₃ | 2-chloro-phenyl | Cl |
| 012 | CH₂CH₃ | 2-chloro-phenyl | OCH₃ |
| 013 | CH₃ | 2-methoxy-phenyl | OH |
| 014 | CH₃ | 2-methoxy-phenyl | Cl |
| 015 | CH₃ | 2-methoxy-phenyl | OCH₃ |
| 016 | CH₂CH₃ | 2-methoxy-phenyl | OH |
| 017 | CH₂CH₃ | 2-methoxy-phenyl | Cl |
| 018 | CH₂CH₃ | 2-methoxy-phenyl | OCH₃ |
| 019 | CH₃ | 2-trifluoromethyl-phenyl | OH |
| 020 | CH₃ | 2-trifluoromethyl-phenyl | Cl |
| 021 | CH₃ | 2-trifluoromethyl-phenyl | OCH₃ |
| 022 | CH₂CH₃ | 2-trifluoromethyl-phenyl | OH |
| 023 | CH₂CH₃ | 2-trifluoromethyl-phenyl | Cl |
| 024 | CH₂CH₃ | 2-trifluoromethyl-phenyl | OCH₃ |
| 025 | CH₃ | 2,4-difluoro-phenyl | OH |
| 026 | CH₃ | 2,4-difluoro-phenyl | Cl |
| 027 | CH₃ | 2,4-difluoro-phenyl | OCH₃ |
| 028 | CH₂CH₃ | 2,4-difluoro-phenyl | OH |
| 029 | CH₂CH₃ | 2,4-difluoro-phenyl | Cl |
| 030 | CH₂CH₃ | 2,4-difluoro-phenyl | OCH₃ |
| 031 | CH₃ | 2,6-difluoro-phenyl | OH |
| 032 | CH₃ | 2,6-difluoro-phenyl | Cl |
| 033 | CH₃ | 2,6-difluoro-phenyl | OCH₃ |
| 034 | CH₂CH₃ | 2,6-difluoro-phenyl | OH |
| 035 | CH₂CH₃ | 2,6-difluoro-phenyl | Cl |
| 036 | CH₂CH₃ | 2,6-difluoro-phenyl | OCH₃ |
| 037 | CH₃ | 2,4-dichloro-phenyl | OH |
| 038 | CH₃ | 2,4-dichioro-phenyl | Cl |
| 039 | CH₃ | 2,4-dichloro-phenyl | OCH₃ |
| 040 | CH₂CH₃ | 2,4-dichloro-phenyl | OH |
| 041 | CH₂CH₃ | 2,4-dichloro-phenyl | Cl |
| 042 | CH₂CH₃ | 2,4-dichloro-phenyl | OCH₃ |
| 043 | CH₃ | 2,6-dichloro-phenyl | OH |
| 044 | CH₃ | 2,6-dichloro-phenyl | Cl |
| 045 | CH₃ | 2,6-dichloro-phenyl | OCH₃ |
| 046 | CH₂CH₃ | 2,6-dichloro-phenyl | OH |
| 047 | CH₂CH₃ | 2,6-dichloro-phenyl | Cl |
| 048 | CH₂CH₃ | 2,6-dichloro-phenyl | OCH₃ |
| 049 | CH₃ | 4-chloro-2-fluoro-phenyl | OH |
| 050 | CH₃ | 4-chloro-2-fluoro-phenyl | Cl |
| 051 | CH₃ | 4-chloro-2-fluoro-phenyl | OCH₃ |
| 052 | CH₂CH₃ | 4-chloro-2-fluoro-phenyl | OH |
| 053 | CH₂CH₃ | 4-chloro-2-fluoro-phenyl | Cl |
| 054 | CH₂CH₃ | 4-chloro-2-fluoro-phenyl | OCH₃ |
| 055 | CH₃ | 2-chloro-4-fluoro-phenyl | OH |
| 056 | CH₃ | 2-chloro-4-fluoro-phenyl | Cl |
| 057 | CH₃ | 2-chloro-4-fluoro-phenyl | OCH₃ |
| 058 | CH₂CH₃ | 2-chloro-4-fluoro-phenyl | OH |
| 059 | CH₂CH₃ | 2-chloro-4-fluoro-phenyl | Cl |
| 060 | CH₂CH₃ | 2-chloro-4-fluoro-phenyl | OCH₃ |
| 061 | CH₃ | 2-chloro-6-fluoro-phenyl | OH |
| 062 | CH₃ | 2-chloro-6-fluoro-phenyl | Cl |
| 063 | CH₃ | 2-chloro-6-fluoro-phenyl | OCH₃ |
| 064 | CH₂CH₃ | 2-chloro-6-fluoro-phenyl | OH |
| 065 | CH₂CH₃ | 2-chloro-6-fluoro-phenyl | Cl |
| 066 | CH₂CH₃ | 2-chloro-6-fluoro-phenyl | OCH₃ |
| 067 | CH₃ | 2-fluoro-4-methoxy-phenyl | OH |
| 068 | CH₃ | 2-fluoro-4-methoxy-phenyl | Cl |
| 069 | CH₃ | 2-fluoro-4-methoxy-phenyl | OCH₃ |
| 070 | CH₂CH₃ | 2-fluoro-4-methoxy-phenyl | OH |
| 071 | CH₂CH₃ | 2-fluoro-4-methoxy-phenyl | Cl |
| 072 | CH₂CH₃ | 2-fluoro-4-methoxy-phenyl | OCH₃ |
| 073 | CH₃ | 2-fluoro-6-methoxy-phenyl | OH |
| 074 | CH₃ | 2-fluoro-6-methoxy-phenyl | Cl |
| 075 | CH₃ | 2-fluoro-6-methoxy-phenyl | OCH₃ |
| 076 | CH₂CH₃ | 2-fluoro-6-methoxy-phenyl | OH |
| 077 | CH₂CH₃ | 2-fluoro-6-methoxy-phenyl | Cl |
| 078 | CH₂CH₃ | 2-fluoro-6-methoxy-phenyl | OCH₃ |
| 079 | CH₃ | 4-fluoro-2-methoxy-phenyl | OH |
| 080 | CH₃ | 4-fluoro-2-methoxy-phenyl | Cl |
| 081 | CH₃ | 4-fluoro-2-methoxy-phenyl | OCH₃ |
| 082 | CH₂CH₃ | 4-fluoro-2-methoxy-phenyl | OH |
| 083 | CH₂CH₃ | 4-fluoro-2-methoxy-phenyl | Cl |
| 084 | CH₂CH₃ | 4-fluoro-2-methoxy-phenyl | OCH₃ |
| 085 | CH₃ | 2-fluoro-4-trifluoromethyl-phenyl | OH |
| 086 | CH₃ | 2-fluoro-4-trifluoromethyl-phenyl | Cl |
| 087 | CH₃ | 2-fluoro-4-trifluoromethyl-phenyl | OCH₃ |
| 088 | CH₂CH₃ | 2-fluoro-4-trifluoromethyl-phenyl | OH |
| 089 | CH₂CH₃ | 2-fluoro-4-trifluoromethyl-phenyl | Cl |
| 090 | CH₂CH₃ | 2-fluoro-4-trifluoromethyl-phenyl | OCH₃ |
| 091 | CH₃ | 2-fluoro-6-trifluoromethyl-phenyl | OH |
| 092 | CH₃ | 2-fluoro-6-trifluoromethyl-phenyl | Cl |
| 093 | CH₃ | 2-fluoro-6-trifluoromethyl-phenyl | OCH₃ |
| 094 | CH₂CH₃ | 2-fluoro-6-trifluoromethyl-phenyl | OH |
| 095 | CH₂CH₃ | 2-fluoro-6-trifluoromethyl-phenyl | Cl |
| 096 | CH₂CH₃ | 2-fluoro-6-trifluoromethyl-phenyl | OCH₃ |
| 097 | CH₃ | 4-fluoro-2-trifluoromethyl-phenyl | OH |
| 098 | CH₃ | 4-fluoro-2-trifluoromethyl-phenyl | Cl |
| 099 | CH₃ | 4-fluoro-2-trifluoromethyl-phenyl | OCH₃ |
| 100 | CH₂CH₃ | 4-fluoro-2-trifluoromethyl-phenyl | OH |
| 101 | CH₂CH₃ | 4-fluoro-2-trifluoromethyl-phenyl | Cl |
| 102 | CH₂CH₃ | 4-fluoro-2-trifluoromethyl-phenyl | OCH₃ |
| 103 | CH₃ | 2-chloro-4-methoxy-phenyl | OH |
| 104 | CH₃ | 2-chloro-4-methoxy-phenyl | Cl |
| 105 | CH₃ | 2-chloro-4-methoxy-phenyl | OCH₃ |
| 106 | CH₂CH₃ | 2-chloro-4-methoxy-phenyl | OH |
| 107 | CH₂CH₃ | 2-chloro-4-methoxy-phenyl | Cl |
| 108 | CH₂CH₃ | 2-chloro-4-methoxy-phenyl | OCH₃ |
| 109 | CH₃ | 2-chloro-6-methoxy-phenyl | OH |
| 110 | CH₃ | 2-chloro-6-methoxy-phenyl | Cl |
| 111 | CH₃ | 2-chloro-6-methoxy-phenyl | OCH₃ |
| 112 | CH₂CH₃ | 2-chloro-6-methoxy-phenyl | OH |
| 113 | CH₂CH₃ | 2-chloro-6-methoxy-phenyl | Cl |
| 114 | CH₂CH₃ | 2-chloro-6-methoxy-phenyl | OCH₃ |
| 115 | CH₃ | 4-chloro-2-methoxy-phenyl | OH |
| 116 | CH₃ | 4-chloro-2-methoxy-phenyl | Cl |
| 117 | CH₃ | 4-chloro-2-methoxy-phenyl | OCH₃ |
| 118 | CH₂CH₃ | 4-chloro-2-methoxy-phenyl | OH |
| 119 | CH₂CH₃ | 4-chloro-2-methoxy-phenyl | Cl |
| 120 | CH₂CH₃ | 4-chloro-2-methoxy-phenyl | OCH₃ |
| 121 | CH₃ | 2-chloro-4-trifluoromethyl-phenyl | OH |
| 122 | CH₃ | 2-chloro-4-trifluoromethyl-phenyl | Cl |
| 123 | CH₃ | 2-chloro-4-trifluoromethyl-phenyl | OCH₃ |
| 124 | CH₂CH₃ | 2-chloro-4-trifluoromethyl-phenyl | OH |
| 125 | CH₂CH₃ | 2-chloro-4-trifluoromethyl-phenyl | Cl |
| 126 | CH₂CH₃ | 2-chloro-4-trifluoromethyl-phenyl | OCH₃ |
| 127 | CH₃ | 2-chloro-6-trifluoromethyl-phenyl | OH |
| 128 | CH₃ | 2-chloro-6-trifluoromethyl-phenyl | Cl |
| 129 | CH₃ | 2-chloro-6-trifluoromethyl-phenyl | OCH₃ |
| 130 | CH₂CH₃ | 2-chloro-6-trifluoromethyl-phenyl | OH |
| 131 | CH₂CH₃ | 2-chloro-6-trifluoromethyl-phenyl | Cl |
| 132 | CH₂CH₃ | 2-chloro-6-trifluoromethyl-phenyl | OCH₃ |
| 133 | CH₃ | 4-chloro-2-trifluoromethyl-phenyl | OH |
| 134 | CH₃ | 4-chloro-2-trifluoromethyl-phenyl | Cl |
| 135 | CH₃ | 4-chloro-2-trifluoromethyl-phenyl | OCH₃ |
| 136 | CH₂CH₃ | 4-chloro-2-trifluoromethyl-phenyl | OH |
| 137 | CH₂CH₃ | 4-chloro-2-trifluoromethyl-phenyl | Cl |
| 138 | CH₂CH₃ | 4-chloro-2-trifluoromethyl-phenyl | OCH₃ |
| 139 | CH₃ | 2,3,4-trifluoro-phenyl | OH |
| 140 | CH₃ | 2,3,4-trifluoro-phenyl | Cl |
| 141 | CH₃ | 2,3,4-trifluoro-phenyl | OCH₃ |
| 142 | CH₂CH₃ | 2,3,4-trifluoro-phenyl | OH |
| 143 | CH₂CH₃ | 2,3,4-trifluoro-phenyl | Cl |
| 144 | CH₂CH₃ | 2,3,4-trifluoro-phenyl | OCH₃ |
| 145 | CH₃ | 2,3,6-trifluoro-phenyl | OH |
| 146 | CH₃ | 2,3,6-trifluoro-phenyl | Cl |
| 147 | CH₃ | 2,3,6-trifluoro-phenyl | OCH₃ |
| 148 | CH₂CH₃ | 2,3,6-trifluoro-phenyl | OH |
| 149 | CH₂CH₃ | 2,3,6-trifluoro-phenyl | Cl |
| 150 | CH₂CH₃ | 2,3,6-trifluoro-phenyl | OCH₃ |
| 151 | CH₃ | 2,4,5-trifluoro-phenyl | OH |
| 152 | CH₃ | 2,4,5-trifluoro-phenyl | Cl |
| 153 | CH₃ | 2,4,5-trifluoro-phenyl | OCH₃ |
| 154 | CH₂CH₃ | 2,4,5-trifluoro-phenyl | OH |
| 155 | CH₂CH₃ | 2,4,5-trifluoro-phenyl | Cl |
| 156 | CH₂CH₃ | 2,4,5-trifluoro-phenyl | OCH₃ |
| 157 | CH₃ | 2,4,6-trifluoro-phenyl | OH |
| 158 | CH₃ | 2,4,6-trifluoro-phenyl | Cl |
| 159 | CH₃ | 2,4,6-trifluoro-phenyl | OCH₃ |
| 160 | CH₂CH₃ | 2,4,6-trifluoro-phenyl | OH |
| 161 | CH₂CH₃ | 2,4,6-trifluoro-phenyl | Cl |
| 162 | CH₂CH₃ | 2,4,6-trifluoro-phenyl | OCH₃ |
| 163 | CH₃ | 2,6-difluoro-4-methoxy-phenyl | OH |
| 164 | CH₃ | 2,6-difluoro-4-methoxy-phenyl | Cl |
| 165 | CH₃ | 2,6-difluoro-4-methoxy-phenyl | OCH₃ |
| 166 | CH₂CH₃ | 2,6-difluoro-4-methoxy-phenyl | OH |
| 167 | CH₂CH₃ | 2,6-difluoro-4-methoxy-phenyl | Cl |
| 168 | CH₂CH₃ | 2,6-difluoro-4-methoxy-phenyl | OCH₃ |
| 169 | CH₃ | 2,4-difluoro-6-methoxy-phenyl | OH |
| 170 | CH₃ | 2,4-difluoro-6-methoxy-phenyl | Cl |
| 171 | CH₃ | 2,4-difluoro-6-methoxy-phenyl | OCH₃ |
| 172 | CH₂CH₃ | 2,4-difluoro-6-methoxy-phenyl | OH |
| 173 | CH₂CH₃ | 2,4-difluoro-6-methoxy-phenyl | Cl |
| 174 | CH₂CH₃ | 2,4-difluoro-6-methoxy-phenyl | OCH₃ |
| 175 | CH₃ | pentafluoro-phenyl | OH |
| 176 | CH₃ | pentafluoro-phenyl | Cl |
| 177 | CH₃ | pentafluoro-phenyl | OCH₃ |
| 178 | CH₂CH₃ | pentafluoro-phenyl | OH |
| 179 | CH₂CH₃ | pentafluoro-phenyl | Cl |
| 180 | CH₂CH₃ | pentafluoro-phenyl | OCH₃ |
| 181 | CH₃ | 3-fluoro-pyridin-2-yl | OH |
| 182 | CH₃ | 3-fluoro-pyridin-2-yl | Cl |
| 183 | CH₃ | 3-fluoro-pyridin-2-yl | OCH₃ |
| 184 | CH₂CH₃ | 3-fluoro-pyridin-2-yl | OH |
| 185 | CH₂CH₃ | 3-fluoro-pyridin-2-yl | Cl |
| 186 | CH₂CH₃ | 3-fluoro-pyridin-2-yl | OCH₃ |
| 187 | CH₃ | 3-chloro-pyridin-2-yl | OH |
| 188 | CH₃ | 3-chloro-pyridin-2-yl | Cl |
| 189 | CH₃ | 3-chloro-pyridin-2-yl | OCH₃ |
| 190 | CH₂CH₃ | 3-chloro-pyridin-2-yl | OH |
| 191 | CH₂CH₃ | 3-chloro-pyridin-2-yl | Cl |
| 192 | CH₂CH₃ | 3-chloro-pyridin-2-yl | OCH₃ |
| 193 | CH₃ | 3-methoxy-pyridin-2-yl | OH |
| 194 | CH₃ | 3-methoxy-pyridin-2-yl | Cl |
| 195 | CH₃ | 3-methoxy-pyridin-2-yl | OCH₃ |
| 196 | CH₂CH₃ | 3-methoxy-pyridin-2-yl | OH |
| 197 | CH₂CH₃ | 3-methoxy-pyridin-2-yl | Cl |
| 198 | CH₂CH₃ | 3-methoxy-pyridin-2-yl | OCH₃ |
| 199 | CH₃ | 3-trifluoromethyl-pyridin-2-yl | OH |
| 200 | CH₃ | 3-trifluoromethyl-pyridin-2-yl | Cl |
| 201 | CH₃ | 3-trifluoromethyl-pyridin-2-yl | OCH₃ |
| 202 | CH₂CH₃ | 3-trifluoromethyl-pyridin-2-yl | OH |
| 203 | CH₂CH₃ | 3-trifluoromethyl-pyridin-2-yl | Cl |
| 204 | CH₂CH₃ | 3-trifluoromethyl-pyridin-2-yl | OCH₃ |
| 205 | CH₃ | 3,5-difluoro-pyridin-2-yl | OH |
| 206 | CH₃ | 3,5-difluoro-pyridin-2-yl | Cl |
| 207 | CH₃ | 3,5-difluoro-pyridin-2-yl | OCH₃ |
| 208 | CH₂CH₃ | 3,5-difluoro-pyridin-2-yl | OH |
| 209 | CH₂CH₃ | 3,5-difluoro-pyridin-2-yl | Cl |
| 210 | CH₂CH₃ | 3,5-difluoro-pyridin-2-yl | OCH₃ |
| 211 | CH₃ | 3,5-dichloro-pyridin-2-yl | OH |
| 212 | CH₃ | 3,5-dichloro-pyridin-2-yl | Cl |
| 213 | CH₃ | 3,5-dichloro-pyridin-2-yl | OCH₃ |
| 214 | CH₂CH₃ | 3,5-dichloro-pyridin-2-yl | OH |
| 215 | CH₂CH₃ | 3,5-dichloro-pyridin-2-yl | Cl |
| 216 | CH₂CH₃ | 3,5-dichloro-pyridin-2-yl | OCH₃ |
| 217 | CH₃ | 3-chloro-5-fluoro-pyridin-2-yl | OH |
| 218 | CH₃ | 3-chloro-5-fluoro-pyridin-2-yl | Cl |
| 219 | CH₃ | 3-chloro-5-fluoro-pyridin-2-yl | OCH₃ |
| 220 | CH₂CH₃ | 3-chloro-5-fluoro-pyridin-2-yl | OH |
| 221 | CH₂CH₃ | 3-chloro-5-fluoro-pyridin-2-yl | Cl |
| 222 | CH₂CH₃ | 3-chloro-5-fluoro-pyridin-2-yl | OCH₃ |
| 223 | CH₃ | 5-chloro-3-fluoro-pyridin-2-yl | OH |
| 224 | CH₃ | 5-chloro-3-fluoro-pyridin-2-yl | Cl |
| 225 | CH₃ | 5-chloro-3-fluoro-pyridin-2-yl | OCH₃ |
| 226 | CH₂CH₃ | 5-chloro-3-fluoro-pyridin-2-yl | OH |
| 227 | CH₂CH₃ | 5-chloro-3-fluoro-pyridin-2-yl | Cl |
| 228 | CH₂CH₃ | 5-chloro-3-fluoro-pyridin-2-yl | OCH₃ |
| 229 | CH₃ | 3-fluoro-5-methoxy-pyridin-2-yl | OH |
| 230 | CH₃ | 3-fluoro-5-methoxy-pyridin-2-yl | Cl |
| 231 | CH₃ | 3-fluoro-5-methoxy-pyridin-2-yl | OCH₃ |
| 232 | CH₂CH₃ | 3-fluoro-5-methoxy-pyridin-2-yl | OH |
| 233 | CH₂CH₃ | 3-fluoro-5-methoxy-pyridin-2-yl | Cl |
| 234 | CH₂CH₃ | 3-fluoro-5-methoxy-pyridin-2-yl | OCH₃ |
| 235 | CH₃ | 5-fluoro-3-methoxy-pyridin-2-yl | OH |
| 236 | CH₃ | 5-fluoro-3-methoxy-pyridin-2-yl | Cl |
| 237 | CH₃ | 5-fluoro-3-methoxy-pyridin-2-yl | OCH₃ |
| 238 | CH₂CH₃ | 5-fluoro-3-methoxy-pyridin-2-yl | OH |
| 239 | CH₂CH₃ | 5-fluoro-3-methoxy-pyridin-2-yl | Cl |
| 240 | CH₂CH₃ | 5-fluoro-3-methoxy-pyridin-2-yl | OCH₃ |
| 241 | CH₃ | 3-fluoro-5-trifluoromethyl-pyridin-2-yl | OH |
| 242 | CH₃ | 3-fluoro-5-trifluoromethyl-pyridin-2-yl | Cl |
| 243 | CH₃ | 3-fluoro-5-trifluoromethyl-pyridin-2-yl | OCH₃ |
| 244 | CH₂CH₃ | 3-fluoro-5-trifluoromethyl-pyridin-2-yl | OH |
| 245 | CH₂CH₃ | 3-fluoro-5-trifluoromethyl-pyridin-2-yl | Cl |
| 246 | CH₂CH₃ | 3-fluoro-5-trifluoromethyl-pyridin-2-yl | OCH₃ |
| 247 | CH₃ | 5-fluoro-3-trifluoromethyl-pyridin-2-yl | OH |
| 248 | CH₃ | 5-fluoro-3-trifluoromethyl-pyridin-2-yl | Cl |
| 249 | CH₃ | 5-fluoro-3-trifluoromethyl-pyridin-2-yl | OCH₃ |
| 250 | CH₂CH₃ | 5-fluoro-3-trifluoromethyl-pyridin-2-yl | OH |
| 251 | CH₂CH₃ | 5-fluoro-3-trifluoromethyl-pyridin-2-yl | Cl |
| 252 | CH₂CH₃ | 5-fluoro-3-trifluoromethyl-pyridin-2-yl | OCH₃ |
| 253 | CH₃ | 3-chloro-5-methoxy-pyridin-2-yl | OH |
| 254 | CH₃ | 3-chloro-5-methoxy-pyridin-2-yl | Cl |
| 255 | CH₃ | 3-chloro-5-methoxy-pyridin-2-yl | OCH₃ |
| 256 | CH₂CH₃ | 3-chloro-5-methoxy-pyridin-2-yl | OH |
| 257 | CH₂CH₃ | 3-chloro-5-methoxy-pyridin-2-yl | Cl |
| 258 | CH₂CH₃ | 3-chloro-5-methoxy-pyridin-2-yl | OCH₃ |
| 259 | CH₃ | 5-chloro-3-methoxy-pyridin-2-yl | OH |
| 260 | CH₃ | 5-chloro-3-methoxy-pyridin-2-yl | Cl |
| 261 | CH₃ | 5-chloro-3-methoxy-pyridin-2-yl | OCH₃ |
| 262 | CH₂CH₃ | 5-chloro-3-methoxy-pyridin-2-yl | OH |
| 263 | CH₂CH₃ | 5-chloro-3-methoxy-pyridin-2-yl | Cl |
| 264 | CH₂CH₃ | 5-chloro-3-methoxy-pyridin-2-yl | OCH₃ |
| 265 | CH₃ | 3-chloro-5-trifluoromethyl-pyridin-2-yl | OH |
| 266 | CH₃ | 3-chloro-5-trifluoromethyl-pyridin-2-yl | Cl |
| 267 | CH₃ | 3-chloro-5-trifluoromethyl-pyridin-2-yl | OCH₃ |
| 268 | CH₂CH₃ | 3-chloro-5-trifluoromethyl-pyridin-2-yl | OH |
| 269 | CH₂CH₃ | 3-chloro-5-trifluoromethyl-pyridin-2-yl | Cl |
| 270 | CH₂CH₃ | 3-chloro-5-trifluoromethyl-pyridin-2-yl | OCH₃ |
| 271 | CH₃ | 5-chloro-3-trifluoromethyl-pyridin-2-yl | OH |
| 272 | CH₃ | 5-chloro-3-trifluoromethyl-pyridin-2-yl | Cl |
| 273 | CH₃ | 5-chloro-3-trifluoromethyl-pyridin-2-yl | OCH₃ |
| 274 | CH₂CH₃ | 5-chloro-3-trifluoromethyl-pyridin-2-yl | OH |
| 275 | CH₂CH₃ | 5-chloro-3-trifluoromethyl-pyridin-2-yl | Cl |
| 276 | CH₂CH₃ | 5-chloro-3-trifluoromethyl-pyridin-2-yl | OCH₃ |
| 277 | CH₃ | 5-chloro-pyrimidin-4-yl | OH |
| 278 | CH₃ | 5-chloro-pyrimidin-4-yl | Cl |
| 279 | CH₃ | 5-chloro-pyrimidin-4-yl | OCH₃ |
| 280 | CH₂CH₃ | 5-chloro-pyrimidin-4-yl | OH |
| 281 | CH₂CH₃ | 5-chloro-pyrimidin-4-yl | Cl |
| 282 | CH₂CH₃ | 5-chloro-pyrimidin-4-yl | OCH₃ |

As shown above, Table 1 provides 282 specific compounds of Formula (I). Structural examples of these compounds are shown below in Formulas (I.a) through (I.aw) wherein R¹, R⁵, and R⁶ are defined in Table 1.

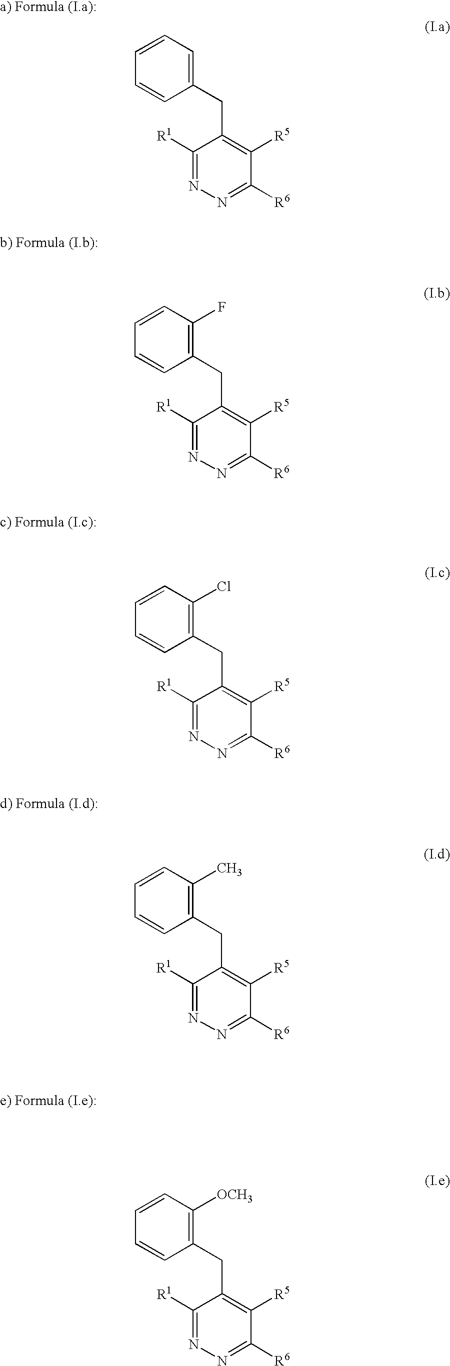

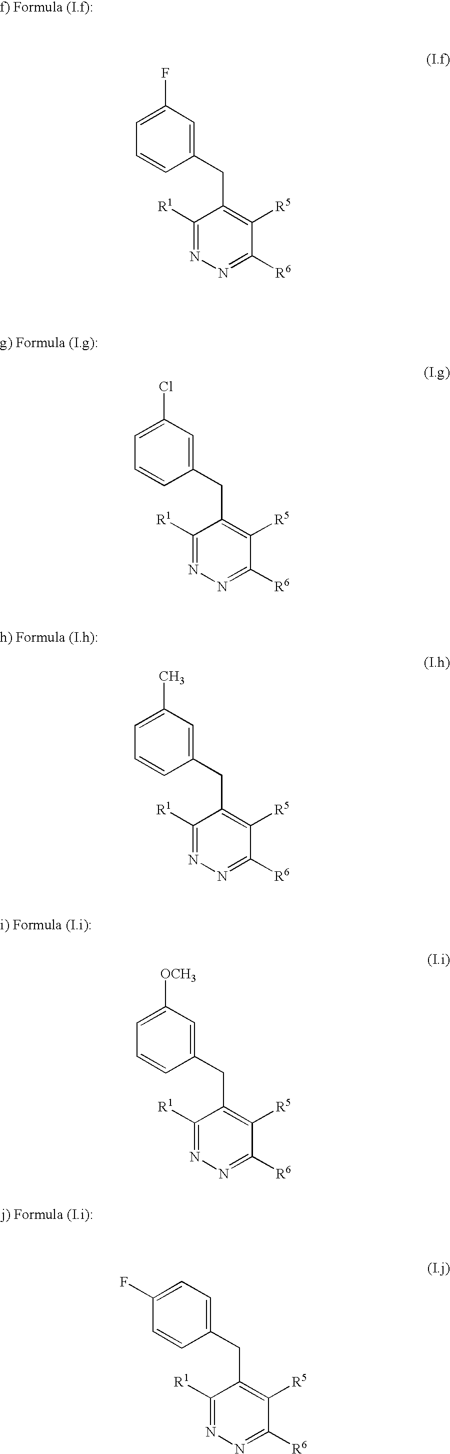

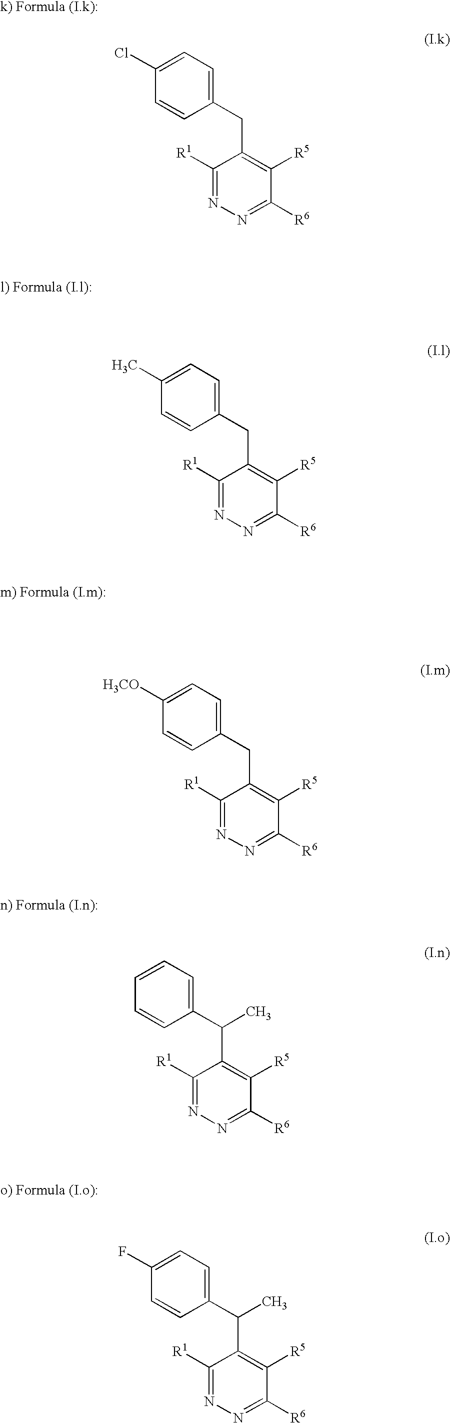

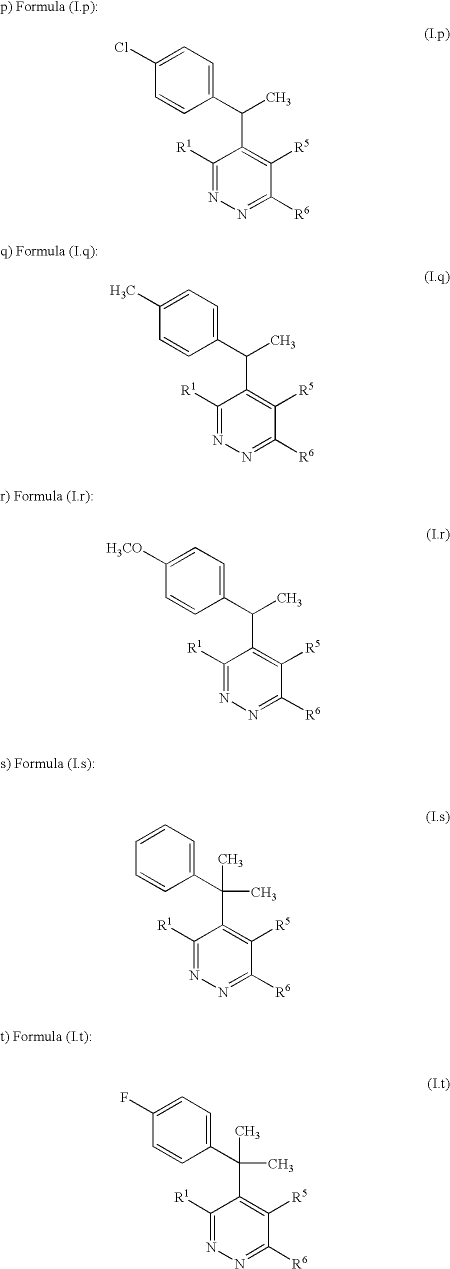

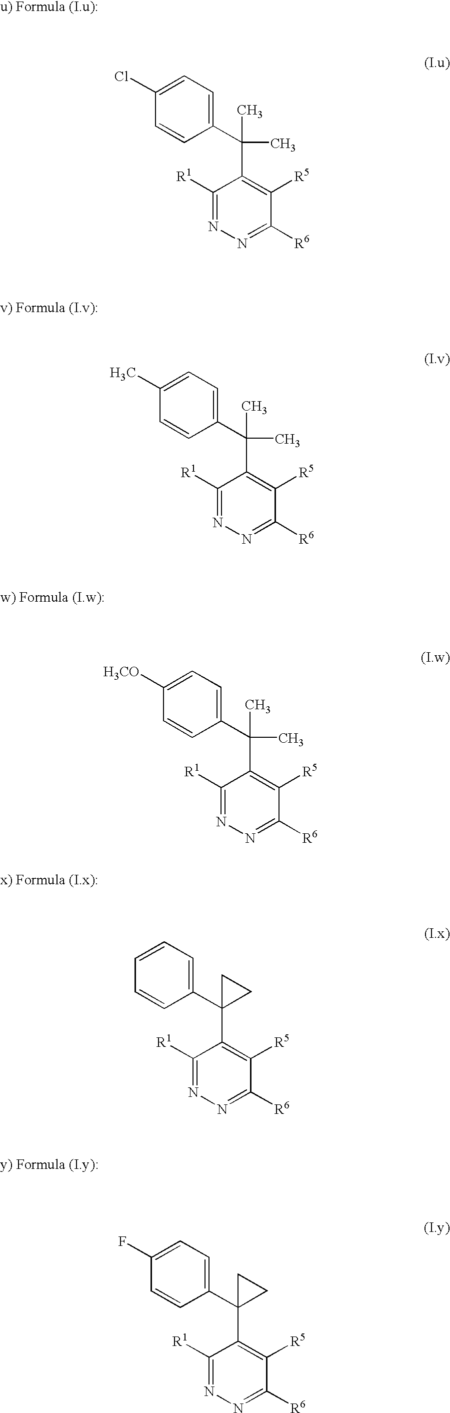

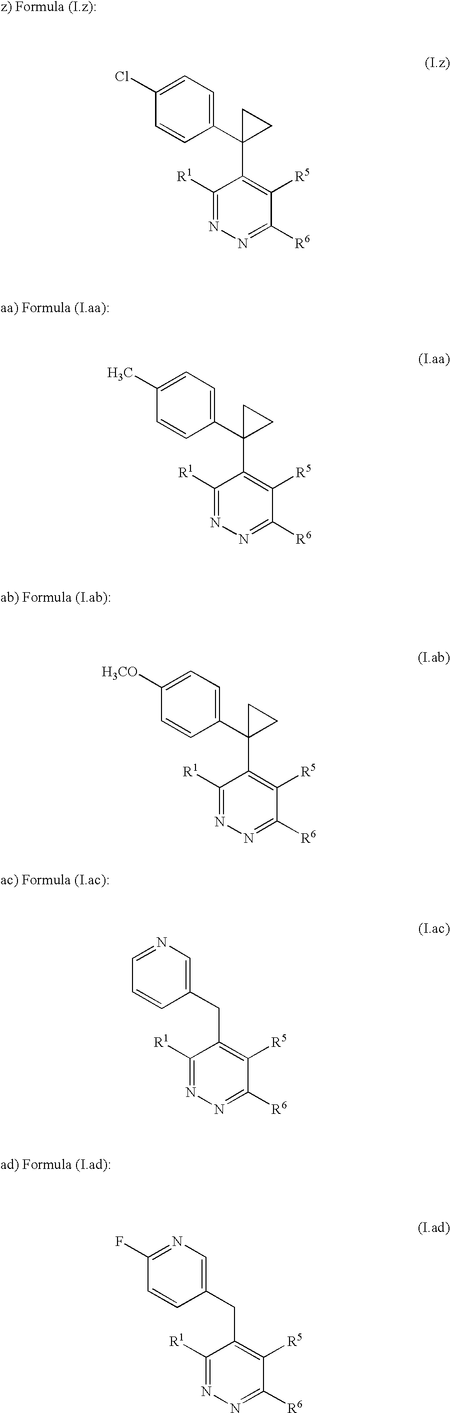

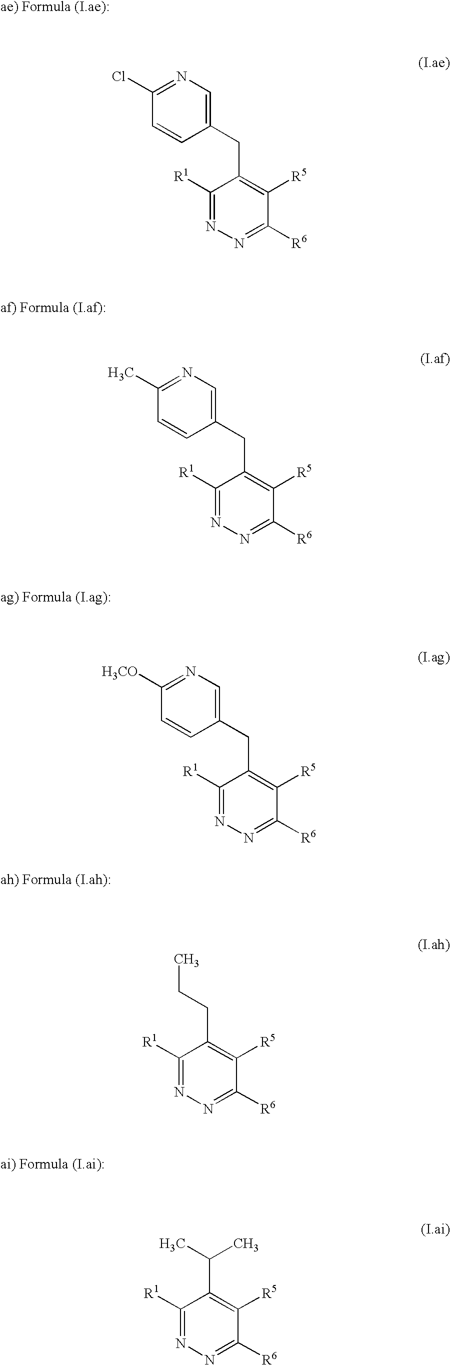

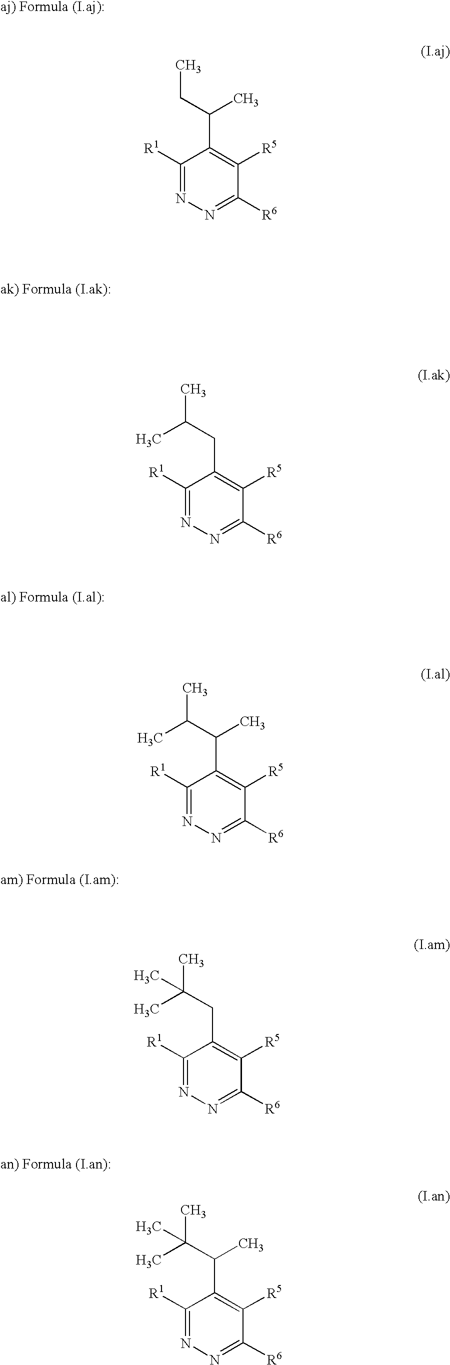

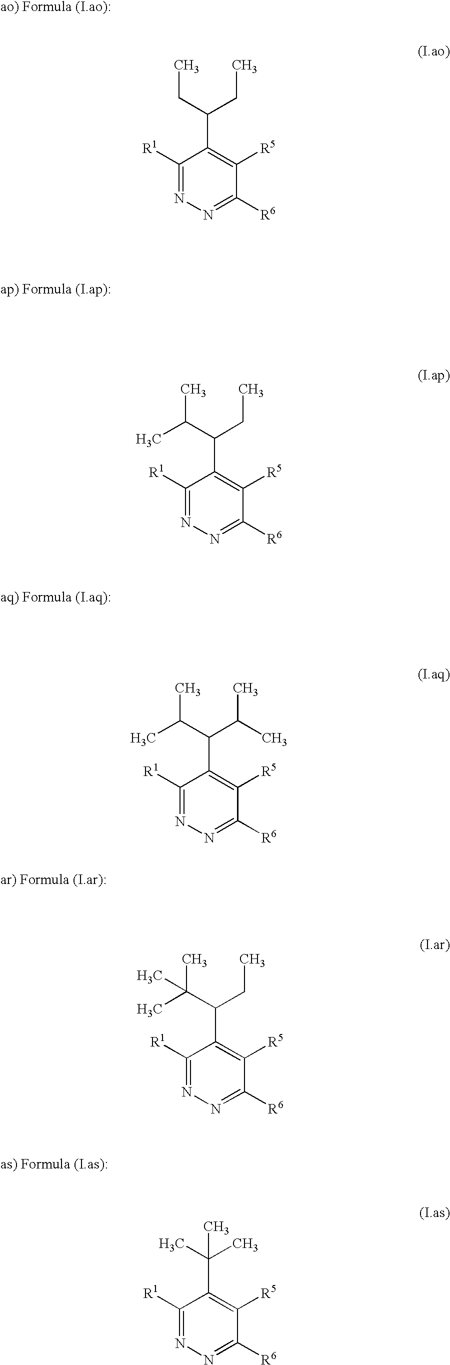

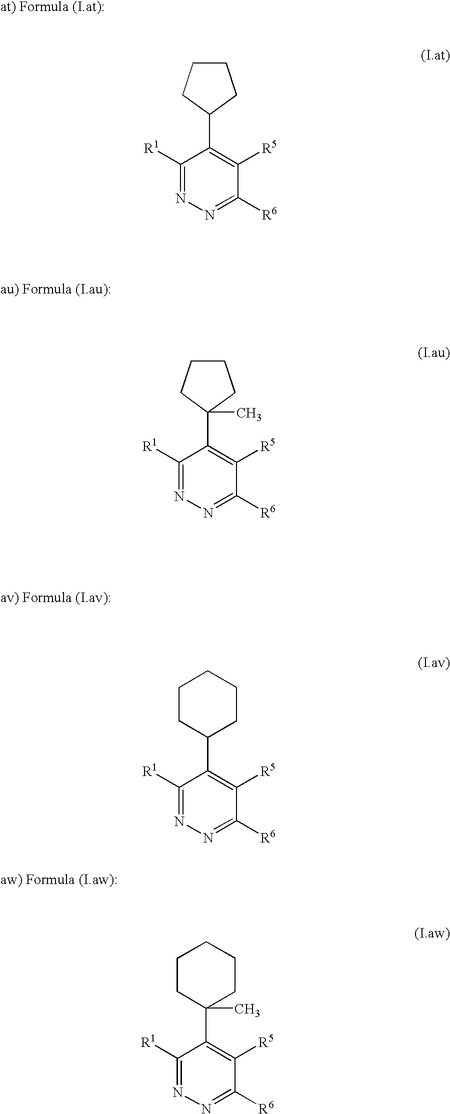

TABLE 2
individual compounds of formula II according to the invention

| Compound | | |
|---|---|---|
| No. | R¹ | R⁵ |
| 01 | CH₃ | 2-fluoro-phenyl |
| 02 | CH₂CH₃ | 2-fluoro-phenyl |
| 03 | CH₃ | 2-chloro-phenyl |
| 04 | CH₂CH₃ | 2-chloro-phenyl |
| 05 | CH₃ | 2-methoxy-phenyl |
| 06 | CH₂CH₃ | 2-methoxy-phenyl |
| 07 | CH₃ | 2-trifluoromethyl-phenyl |
| 08 | CH₂CH₃ | 2-trifluoromethyl-phenyl |
| 09 | CH₃ | 2,4-difluoro-phenyl |
| 10 | CH₂CH₃ | 2,4-difluoro-phenyl |
| 11 | CH₃ | 2,6-difluoro-phenyl |
| 12 | CH₂CH₃ | 2,6-difluoro-phenyl |
| 13 | CH₃ | 2,4-dichloro-phenyl |
| 14 | CH₂CH₃ | 2,4-dichloro-phenyl |
| 15 | CH₃ | 2,6-dichloro-phenyl |
| 16 | CH₂CH₃ | 2,6-dichloro-phenyl |
| 17 | CH₃ | 4-chloro-2-fluoro-phenyl |
| 18 | CH₂CH₃ | 4-chloro-2-fluoro-phenyl |
| 19 | CH₃ | 2-chloro-4-fluoro-phenyl |
| 20 | CH₂CH₃ | 2-chloro-4-fluoro-phenyl |
| 21 | CH₃ | 2-chloro-6-fluoro-phenyl |
| 22 | CH₂CH₃ | 2-chloro-6-fluoro-phenyl |
| 23 | CH₃ | 2-fluoro-4-methoxy-phenyl |
| 24 | CH₂CH₃ | 2-fluoro-4-methoxy-phenyl |
| 25 | CH₃ | 2-fluoro-6-methoxy-phenyl |
| 26 | CH₂CH₃ | 2-fluoro-6-methoxy-phenyl |
| 27 | CH₃ | 4-fluoro-2-methoxy-phenyl |
| 28 | CH₂CH₃ | 4-fluoro-2-methoxy-phenyl |
| 29 | CH₃ | 2-fluoro-4-trifluoromethyl-phenyl |
| 30 | CH₂CH₃ | 2-fluoro-4-trifluoromethyl-phenyl |
| 31 | CH₃ | 2-fluoro-6-trifluoromethyl-phenyl |
| 32 | CH₂CH₃ | 2-fluoro-6-trifluoromethyl-phenyl |
| 33 | CH₃ | 4-fluoro-2-trifluoromethyl-phenyl |
| 34 | CH₂CH₃ | 4-fluoro-2-trifluoromethyl-phenyl |
| 35 | CH₃ | 2-chloro-4-methoxy-phenyl |
| 36 | CH₂CH₃ | 2-chloro-4-methoxy-phenyl |
| 37 | CH₃ | 2-chloro-6-methoxy-phenyl |
| 38 | CH₂CH₃ | 2-chloro-6-methoxy-phenyl |
| 39 | CH₃ | 4-chloro-2-methoxy-phenyl |
| 40 | CH₂CH₃ | 4-chloro-2-methoxy-phenyl |
| 41 | CH₃ | 2-chloro-4-trifluoromethyl-phenyl |
| 42 | CH₂CH₃ | 2-chloro-4-trifluoromethyl-phenyl |
| 43 | CH₃ | 2-chloro-6-trifluoromethyl-phenyl |
| 44 | CH₂CH₃ | 2-chloro-6-trifluoromethyl-phenyl |
| 45 | CH₃ | 4-chloro-2-trifluoromethyl-phenyl |
| 46 | CH₂CH₃ | 4-chloro-2-trifluoromethyl-phenyl |
| 47 | CH₃ | 2,3,4-trifluoro-phenyl |
| 48 | CH₂CH₃ | 2,3,4-trifluoro-phenyl |
| 49 | CH₃ | 2,3,6-trifluoro-phenyl |
| 50 | CH₂CH₃ | 2,3,6-trifluoro-phenyl |
| 51 | CH₃ | 2,4,5-trifluoro-phenyl |
| 52 | CH₂CH₃ | 2,4,5-trifluoro-phenyl |
| 53 | CH₃ | 2,4,6-trifluoro-phenyl |
| 54 | CH₂CH₃ | 2,4,6-trifluoro-phenyl |
| 55 | CH₃ | 2,6-difluoro-4-methoxy-phenyl |
| 56 | CH₂CH₃ | 2,6-difluoro-4-methoxy-phenyl |
| 57 | CH₃ | 2,4-difluoro-6-methoxy-phenyl |
| 58 | CH₂CH₃ | 2,4-difluoro-6-methoxy-phenyl |
| 59 | CH₃ | pentafluoro-phenyl |
| 60 | CH₂CH₃ | pentafluoro-phenyl |
| 61 | CH₃ | 3-fluoro-pyridin-2-yl |
| 62 | CH₂CH₃ | 3-fluoro-pyridin-2-yl |
| 63 | CH₃ | 3-chloro-pyridin-2-yl |
| 64 | CH₂CH₃ | 3-chloro-pyridin-2-yl |
| 65 | CH₃ | 3-methoxy-pyridin-2-yl |
| 66 | CH₂CH₃ | 3-methoxy-pyridin-2-yl |
| 67 | CH₃ | 3-trifluoromethyl-pyridin-2-yl |
| 68 | CH₂CH₃ | 3-trifluoromethyl-pyridin-2-yl |
| 69 | CH₃ | 3,5-difluoro-pyridin-2-yl |
| 70 | CH₂CH₃ | 3,5-difluoro-pyridin-2-yl |
| 71 | CH₃ | 3,5-dichloro-pyridin-2-yl |
| 72 | CH₂CH₃ | 3,5-dichloro-pyridin-2-yl |
| 73 | CH₃ | 3-chloro-5-fluoro-pyridin-2-yl |
| 74 | CH₂CH₃ | 3-chloro-5-fluoro-pyridin-2-yl |
| 75 | CH₃ | 5-chloro-3-fluoro-pyridin-2-yl |
| 76 | CH₂CH₃ | 5-chloro-3-fluoro-pyridin-2-yl |
| 77 | CH₃ | 3-fluoro-5-methoxy-pyridin-2-yl |
| 78 | CH₂CH₃ | 3-fluoro-5-methoxy-pyridin-2-yl |
| 79 | CH₃ | 5-fluoro-3-methoxy-pyridin-2-yl |
| 80 | CH₂CH₃ | 5-fluoro-3-methoxy-pyridin-2-yl |
| 81 | CH₃ | 3-fluoro-5-trifluoromethyl-pyridin-2-yl |
| 82 | CH₂CH₃ | 3-fluoro-5-trifluoromethyl-pyridin-2-yl |
| 83 | CH₃ | 5-fluoro-3-trifluoromethyl-pyridin-2-yl |
| 84 | CH₂CH₃ | 5-fluoro-3-trifluoromethyl-pyridin-2-yl |
| 85 | CH₃ | 3-chloro-5-methoxy-pyridin-2-yl |
| 86 | CH₂CH₃ | 3-chloro-5-methoxy-pyridin-2-yl |
| 87 | CH₃ | 5-chloro-3-methoxy-pyridin-2-yl |
| 88 | CH₂CH₃ | 5-chloro-3-methoxy-pyridin-2-yl |
| 89 | CH₃ | 3-chloro-5-trifluoromethyl-pyridin-2-yl |
| 90 | CH₂CH₃ | 3-chloro-5-trifluoromethyl-pyridin-2-yl |
| 91 | CH₃ | 5-chloro-3-trifluoromethyl-pyridin-2-yl |
| 92 | CH₂CH₃ | 5-chloro-3-trifluoromethyl-pyridin-2-yl |
| 93 | CH₃ | 5-chloro-pyrimidin-4-yl |
| 94 | CH₂CH₃ | 5-chloro-pyrimidin-4-yl |

As shown above, Table 2 provides 94 specific compounds of Formula (II). Structural examples of these compounds are shown below in Formulas (II.a) through (II.aw) wherein R¹ and R⁵ are defined in Table 2.

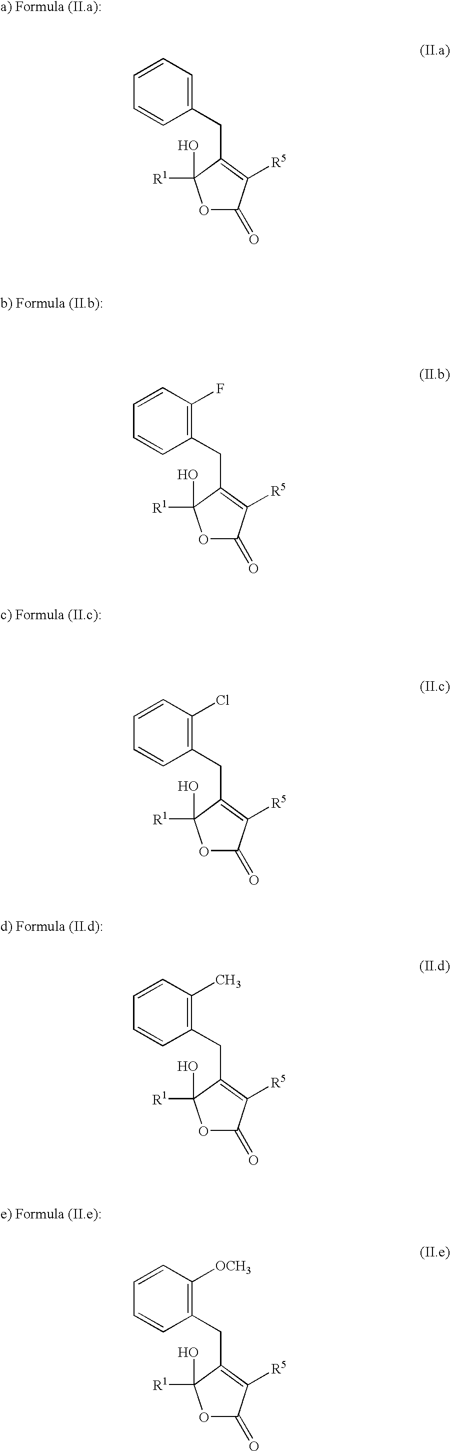

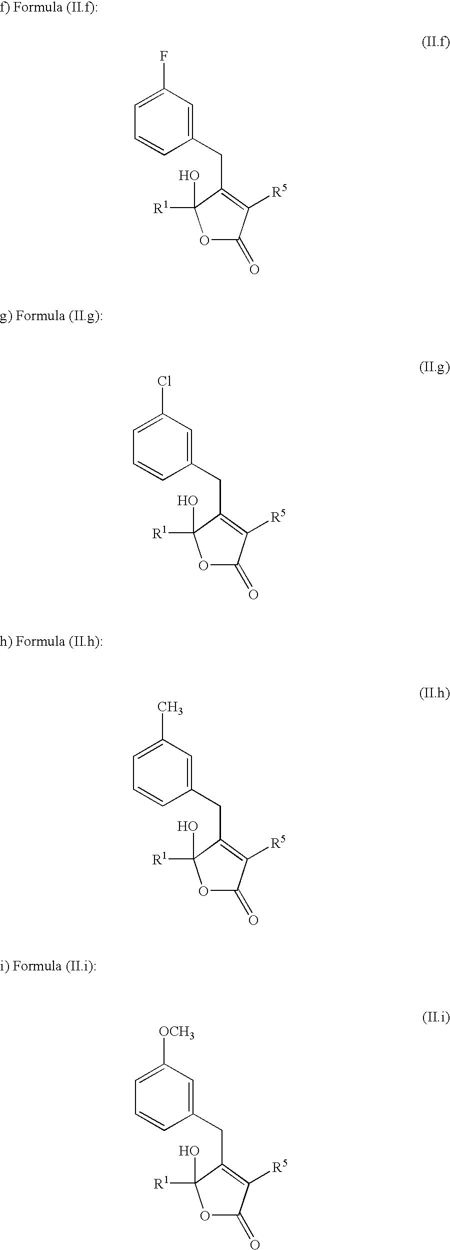

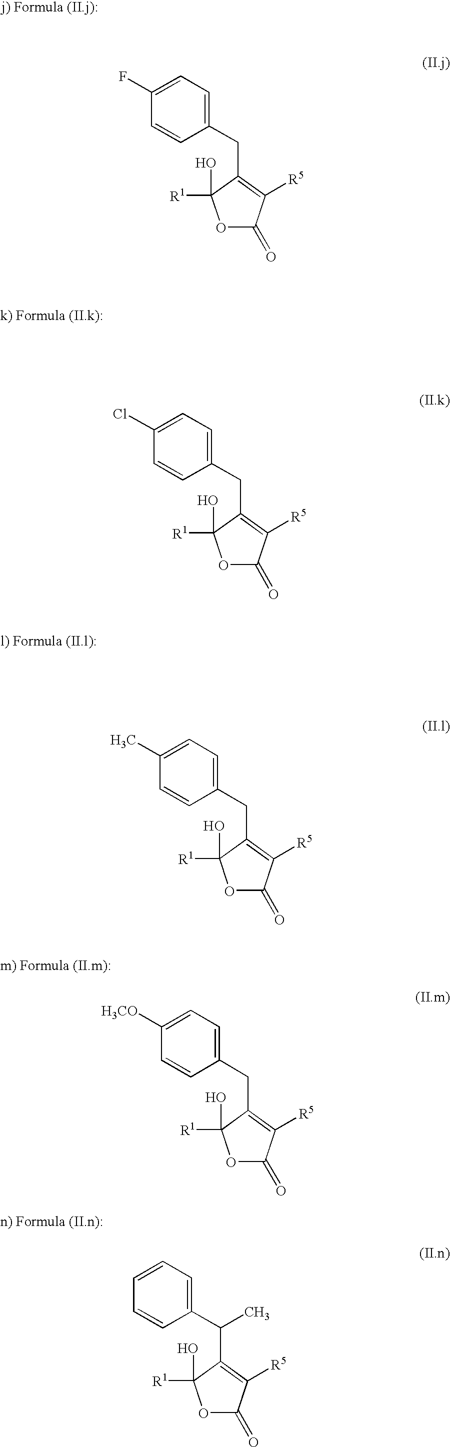

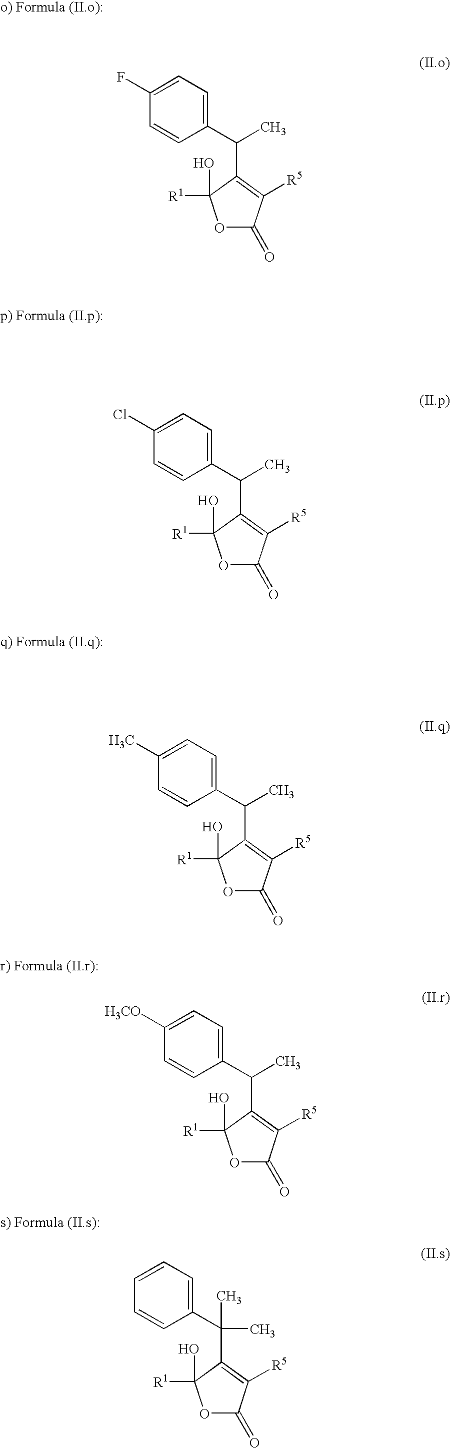

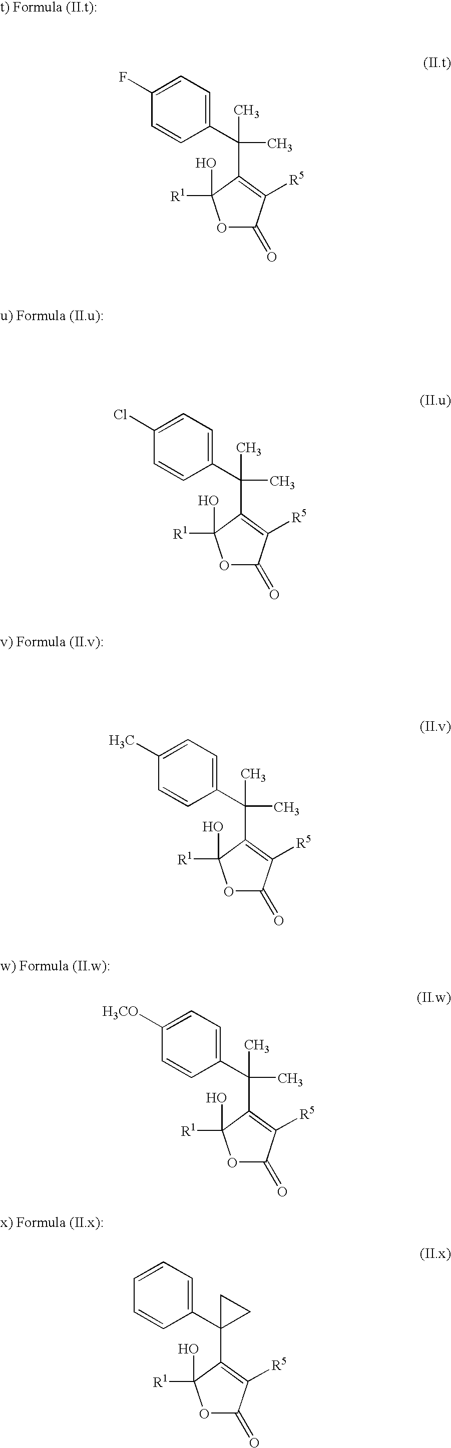

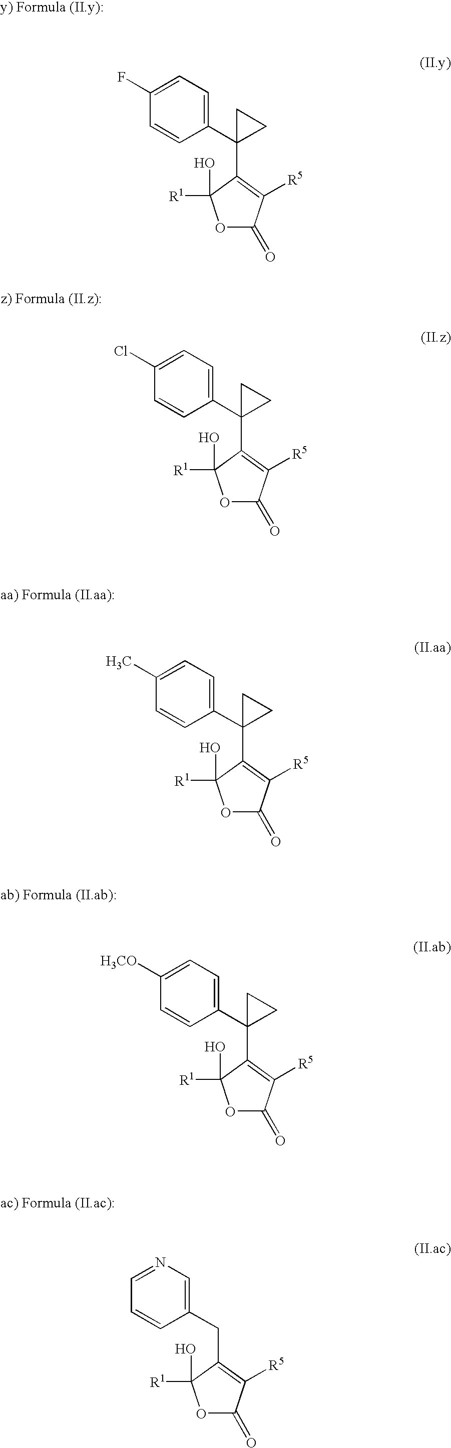

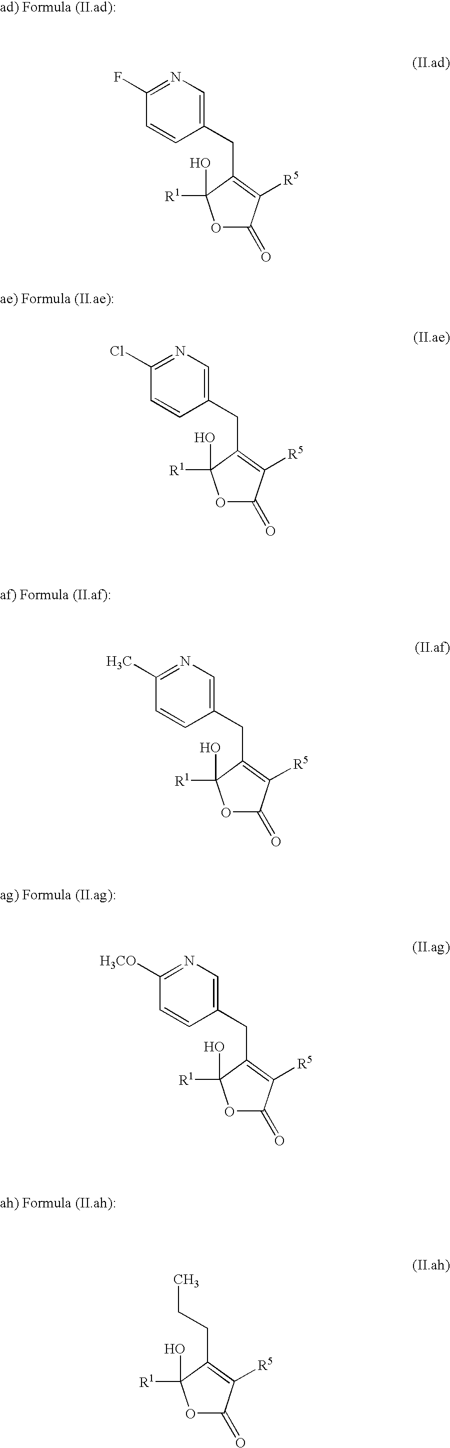

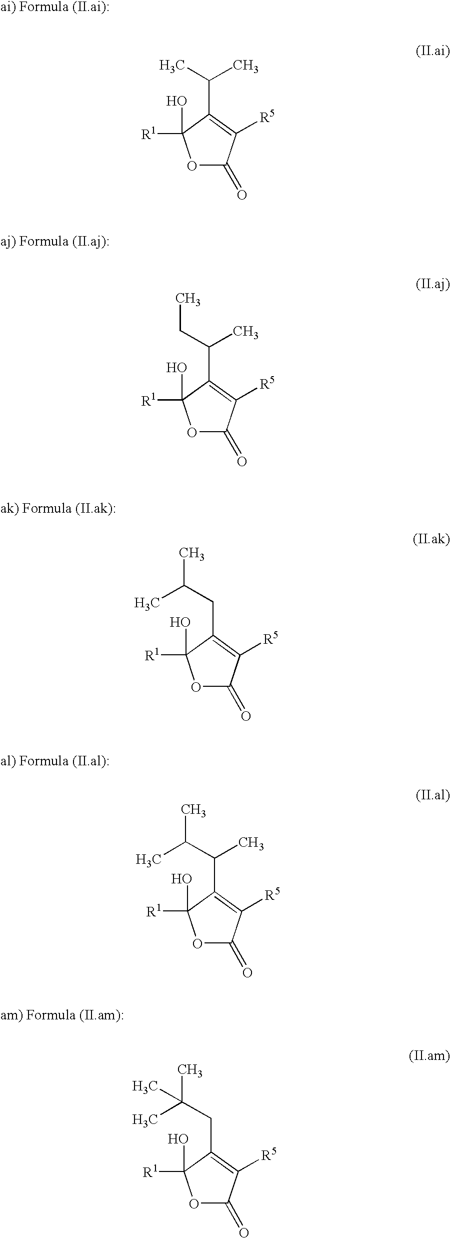

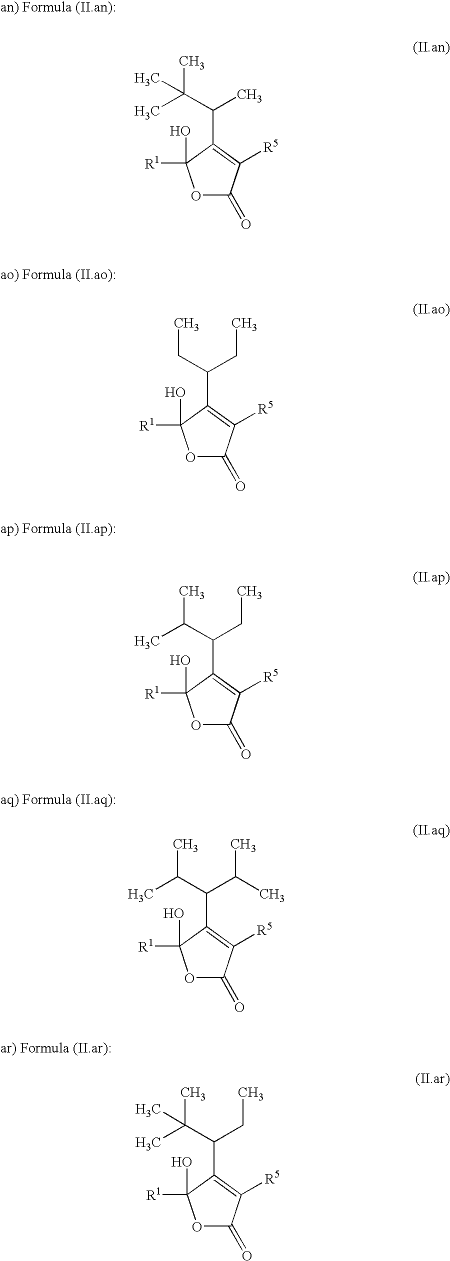

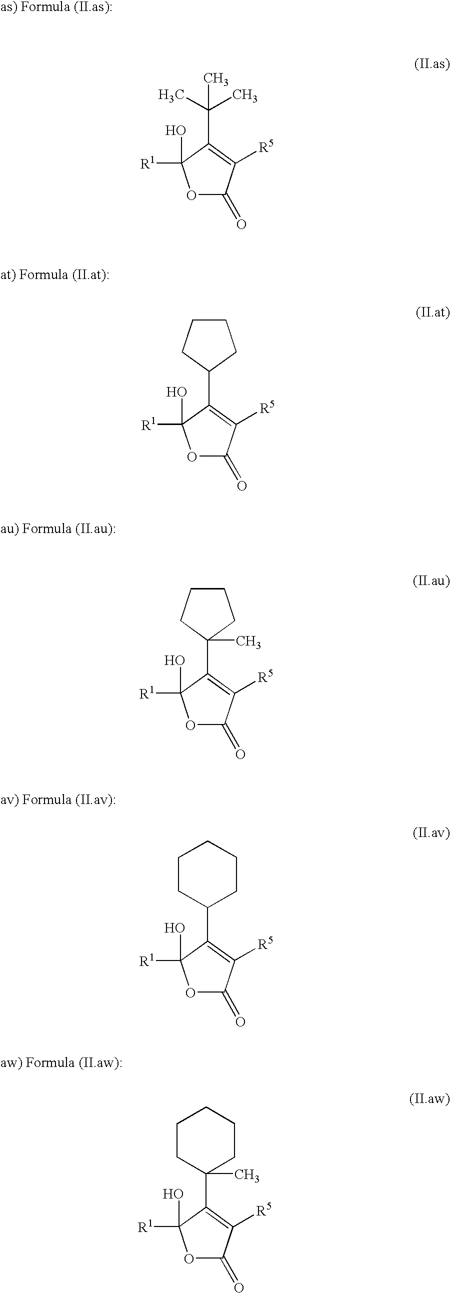

Throughout this description, temperatures are given in degrees Celsius; “NMR” means nuclear magnetic resonance spectrum; and “%” is percent by weight, unless corresponding concentrations are indicated in other units.

The following abbreviations are used throughout this description:

| | |
|---|---|
| m.p. = melting point | br = broad |
| s = singlet | dd = doublet of doublets |
| d = doublet | dt = doublet of triplets |
| t = triplet | q = quartet |
| m = multiplet | ppm = parts per million |

Table 3 shows selected melting point and selected NMR data, all with CDCl₃ as the solvent (unless otherwise stated, no attempt is made to list all characterising data in all cases) for compounds of Tables 1 and 2.

TABLE 3
Melting point and selected NMR data for compounds of Tables 1 and 2

| Compound | ¹H-NMR data | |
|---|---|---|
| Number | (ppm/multiplicity/number of Hs) | m.p. (° C.) |
| I.a.158 | | 106-108 |
| I.c.157 | | 176-180 |
| I.d.157 | | 177-182 |
| I.g.157 | | 196-201 |
| I.h.157 | | 175-180 |
| I.h.158 | | 91-95 |
| I.I.157 | | 222-225 |
| I.I.158 | | 128-131 |
| I.j.157 | | 195-200 |
| I.j.158 | | 70-73 |
| I.k.157 | | 212-215 |
| I.k.158 | 2.62 (s, 3H), 3.86 (s, 2H), | |
| | 6.73-6.81 (m, 4H), | |
| | 7.20 (d, 2H) | |
| I.z.157 | | 250-256 |
| I.z.158 | | 140-144 |
| II.c.53 | | 161-166 |
| II.h.53 | | 166-168 |
| II.k.53 | | 127-129 |
| II.z.53 | | 172-175 |

The compounds according to the present invention can be prepared according to the above-mentioned reaction schemes, in which, unless otherwise stated, the definition of each variable is as defined above for a compound of formula (I).

### Biological Examples

### Alternaria solani/Tomato/Preventive (Action Against Alternaria on Tomato)

4 weeks old tomato plants cv. Roter Gnom are treated with the formulated test compound in a spray chamber. Two days after application tomato plants are inoculated by spraying a spore suspension on the test plants. After an incubation period of 4 days at 22° C./18° C. and 95% r.h. in a greenhouse the disease incidence is assessed.

Compounds I.a.158, I.I.158, I.j.158 and I.k.158 according to the invention at 200 ppm inhibit fungal infestation in this test to at least 80%, while under the same conditions untreated control plants are infected by the phytopathogenic fungi to over 80%.

Botrytis cinerea/tomato/preventive (Action against Botrytis on tomato)

4 weeks old tomato plants cv. Roter Gnom are treated with the formulated test compound in a spray chamber. Two days after application tomato plants are inoculated by spraying a spore suspension on the test plants. After an incubation period of 3 days at 20° C. and 95% r. h. in a greenhouse the disease incidence is assessed.

Compounds I.a.158, I.j.158, I.k.158 and I.z.158 according to the invention at 200 ppm inhibit fungal infestation in this test to at least 80%, while under the same conditions untreated control plants are infected by the phytopathogenic fungi to over 80%.

Puccinia recondita/Wheat/Preventive (Action Against Brown Rust on Wheat)

1 week old wheat plants cv. Arina are treated with the formulated test compound in a spray chamber. One day after application wheat plants are inoculated by spraying a spore suspension (1×105 uredospores/ml) on the test plants. After an incubation period of 1 day at 20° C. and 95% r.h. plants are kept for 10 days 20° C./18° C. (day/night) and 60% r.h. in a greenhouse. The disease incidence is assessed 11 days after inoculation.

Compounds I.a.158, 1.1.158, I.k.158 and I.z.158 according to the invention at 200 ppm inhibit fungal infestation in this test to at least 80%, while under the same conditions untreated control plants are infected by the phytopathogenic fungi to over 80%.

Maqnaporthe qrisea (Pyricularia oryzae)/Rice/Preventive (Action Against Rice Blast)

3 weeks old rice plants cv. Koshihikari are treated with the formulated test compound in a spray chamber. Two days after application rice plants are inoculated by spraying a spore suspension (1×10⁵ conidia/ml) on the test plants. After an incubation period of 6 days at 25° C. and 95% r.h. the disease incidence is assessed.

Compounds I.a.158, I.j.158, I.k.158 and I.z.158 according to the invention at 200 ppm inhibit fungal infestation in this test to at least 80%, while under the same conditions untreated control plants are infected by the phytopathogenic fungi to over 80%.

Pyrenophora teres (Helminthosporium teres)/Barley/Preventive (Action Against Net Blotch on Barley)

1-week-old barley plants cv. Regina are treated with the formulated test compound in a spray chamber. Two days after application barley plants are inoculated by spraying a spore suspension (2.6×10⁴ conidia/ml) on the test plants. After an incubation period of 4 days at 20° C. and 95% r.h. the disease incidence is assessed.

Compounds I.a.158, I.j.158 and I.k.158 according to the invention at 200 ppm inhibit fungal infestation in this test to at least 80%, while under the same conditions untreated control plants are infected by the phytopathogenic fungi to over 80%.

Septoria tritici/Wheat/Preventive (Action Against Septoria Leaf Spot on Wheat)

2 weeks old wheat plants cv. Riband are treated with the formulated test compound in a spray chamber. One day after application wheat plants are inoculated by spraying a spore suspension (10⁶ conidia/ml) on the test plants. After an incubation period of 1 day at 22° C./21° C. and 95% r.h. plants are kept at 22° C./21° C. and 70% r.h. in a greenhouse. The disease incidence is assessed 16-18 days after inoculation.

Compounds I.j.158, I.I.158, I.k.158 and I.z.158 according to the invention at 200 ppm inhibits fungal infestation in this test to at least 80%, while under the same conditions untreated control plants are infected by the phytopathogenic fungi to over 80%.

Uncinula necator/Grape/Preventive (Action Against Powdery Mildew on Grape)

5 weeks old grape seedlings cv. Gutedel are treated with the formulated test compound in a spray chamber. One day after application grape plants are inoculated by shaking plants infected with grape powdery mildew above the test plants. After an incubation period of 7 days at 24° C./22° C. and 70% r.h. under a light regime of 14/10 h (light/dark) the disease incidence is assessed.

Compounds I.a.158, I.j.158, I.k.158 and I.z.158 according to the invention at 200 ppm inhibit fungal infestation in this test to at least 80%, while under the same conditions untreated control plants are infected by the phytopathogenic fungi to over 80%.

1. A compound of formula I: 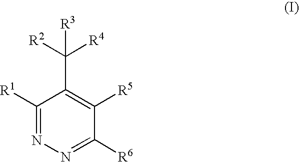
wherein
R¹ is C₁-C₆alkyl, C₁-C₆haloalkyl or C₃-C₆cycloalkyl;
R² is hydrogen or an optionally substituted alkyl, aryl or heteroaryl;
R³ is hydrogen, C₁-C₆alkyl or C₁-C₆haloalkyl;
R⁴ is hydrogen, C₁-C₆alkyl or C₁-C₆haloalkyl; or
R³ and R⁴ together can be part of a carbocyclic or heterocyclic 3- to 8-membered ring;
R⁵ is optionally substituted aryl or heteroaryl; and
R⁶ is hydroxy, halogen, C₁-C₆alkoxy, C₁-C₆haloalkoxy, C₁-C₆alkylthio or C₁-C₆haloalkylthio;
or an agrochemically usable salt form thereof.

2. The compound according to claim 1 wherein R¹ is C₁-C₅alkyl, C₁-C₅haloalkyl or C₃-C₅cycloalkyl.

3. The compound according to claim 1 wherein R² is hydrogen or an optionally substituted C₁-C₆alkyl, phenyl, naphthyl, furyl, thienyl, pyridinyl or quinolinyl.

4. The compound according to claim 1 wherein R³ is hydrogen, C₁-C₅alkyl or C₁-C₅haloalkyl.

5. The compound according to claim 1 wherein R⁴ is hydrogen, C₁-C₅alkyl or C₁-C₅haloalkyl.

6. The compound according to claim 1 wherein R³ and R⁴ together can be part of a carbocyclic or heterocyclic 3- to 7-membered ring.

7. The compound according claim 1 wherein R⁵ is optionally substituted phenyl, pyridinyl, pyrimidinyl, thienyl or thiazolyl.

8. The compound according to claim 1 wherein R⁶ is hydroxy, halogen, C₁-C₄alkoxy, C₁-C₄haloalkoxy, C₁-C₄alkylthio or C₁-C₄haloalkylthio.

9. The compound according to claim 1 wherein
R¹ is C₁-C₄alkyl, C₁-C₄haloalkyl or C₃-C₄cycloalkyl;
R² is hydrogen or an optionally substituted C₁-C₄alkyl, phenyl, furyl, thienyl, pyridinyl or quinolinyl;
R³ is hydrogen or C₁-C₅alkyl;
R⁴ is hydrogen or C₁-C₅alkyl; or
R³ and R⁴ together can be part of a carbocyclic 3- to 7-membered ring;
R⁵ is optionally substituted phenyl, pyridinyl, pyrimidinyl or thienyl; and
R⁶ is hydroxy, halogen, C₁-C₄alkoxy, C₁-C₄haloalkoxy or C₁-C₄alkylthio.

10. The compound according to claim 1 wherein
R¹ is C₁-C₄alkyl, C₁-C₃haloalkyl;
R² is hydrogen or an optionally substituted C₁-C₄alkyl, phenyl, furyl, thienyl or pyridinyl;
R³ is hydrogen or C₁-C₄alkyl;
R⁴ is hydrogen or C₁-C₄alkyl; or
R³ and R⁴ together can be part of a carbocyclic 3- to 6-membered ring;
R⁵ is 2-fluoro-phenyl, 2-chloro-phenyl, 2-methoxy-phenyl, 2-trifluoromethyl-phenyl, 2,4-difluoro-phenyl, 2,6-difluoro-phenyl, 2,4-dichloro-phenyl, 2,6-dichloro-phenyl, 4-chloro-2-fluoro-phenyl, 2-chloro-4-fluoro-phenyl, 2-chloro-6-fluoro-phenyl, 2-fluoro-4-methoxy-phenyl, 2-fluoro-6-methoxy-phenyl, 4-fluoro-2-methoxy-phenyl, 2-fluoro-4-trifluoromethyl-phenyl, 2-fluoro-6-trifluoromethyl-phenyl, 4-fluoro-2-trifluoromethyl-phenyl, 2-chloro-4-methoxy-phenyl, 2-chloro-6-methoxy-phenyl, 4-chloro-2-methoxy-phenyl, 2-chloro-4-trifluoromethyl-phenyl, 2-chloro-6-trifluoromethyl-phenyl, 4-chloro-2-trifluoromethyl-phenyl, 2,3,4-trifluoro-phenyl, 2,3,6-trifluoro-phenyl, 2,4,5-trifluoro-phenyl, 2,4,6-trifluoro-phenyl, 2,6-difluoro-4-methoxy-phenyl, 2,4-difluoro-6-methoxy-phenyl, pentafluoro-phenyl, 3-fluoro-pyridin-2-yl, 3-chloro-pyridin-2-yl, 3-methoxy-pyridin-2-yl, 3-trifluoromethyl-pyridin-2-yl, 3,5-difluoro-pyridin-2-yl, 3,5-dichloro-pyridin-2-yl, 3-chloro-5-fluoro-pyridin-2-yl, 5-chloro-3-fluoro-pyridin-2-yl, 3-fluoro-5-methoxy-pyridin-2-yl, 5-fluoro-3-methoxy-pyridin-2-yl, 3-fluoro-5-trifluoromethyl-pyridin-2-yl, 5-fluoro-3-trifluoromethyl-pyridin-2-yl, 3-chloro-5-methoxy-pyridin-2-yl, 5-chloro-3-methoxy-pyridin-2-yl, 3-chloro-5-trifluoromethyl-pyridin-2-yl, 5-chloro-3-trifluoromethyl-pyridin-2-yl or 5-chloro-pyrimidin-4-yl; and
R⁶ is hydroxy, halogen, C₁-C₃alkoxy or C₁-C₃haloalkoxy.

11. The compound according to claim 1 wherein
R¹ is methyl, ethyl, isopropyl, tertiobutyl or trifluoromethyl;
R² is hydrogen or an optionally substituted C₁-C₄alkyl, phenyl, thienyl or pyridinyl;
R³ is hydrogen, methyl or ethyl;
R⁴ is hydrogen, methyl or ethyl; or
R³ and R⁴ together can be part of a carbocyclic 3- to 5-membered ring;
R⁵ is 2,4-difluoro-phenyl, 2,4-dichloro-phenyl, 2-chloro-6-fluoro-phenyl, 4-fluoro-2-methoxy-phenyl, 2-chloro-4-methoxy-phenyl, 2,4,5-trifluoro-phenyl, 2,4,6-trifluoro-phenyl, 2,6-difluoro-4-methoxy-phenyl, 2,4-difluoro-6-methoxy-phenyl, 3-trifluoromethyl-pyridin-2-yl, 3,5-dichloro-pyridin-2-yl or 5-chloro-pyrimidin-4-yl; and
R⁶ is hydroxy, chloro, fluoro, methoxy, ethoxy or trifluoromethoxy.

12. The compound according to claim 1 wherein
R¹ is methyl;
R² is optionally substituted phenyl;
R³ is hydrogen;
R⁴ is hydrogen; or
R³ and R⁴ together can be part of a carbocyclic 3-membered ring;
R⁵ is 2,4,6-trifluoro-phenyl; and
R⁶ is hydroxy or chloro.

13. A compound selected from
5-benzyl-6-methyl-4-(2,4,6-trifluoro-phenyl)-pyridazin-3-ol;
5-(4-fluoro-benzyl)-6-methyl-4-(2,4,6-trifluoro-phenyl)-pyridazin-3-ol;
5-(4-chloro-benzyl)-6-methyl-4-(2,4,6-trifluoro-phenyl)-pyridazin-3-ol;
3-chloro-5-(2-chloro-benzyl)-6-methyl-4-(2,4,6-trifluoro-phenyl)-pyridazine;
3-chloro-6-methyl-5-(2-methyl-benzyl)-4-(2,4,6-trifluoro-phenyl)-pyridazine;
3-chloro-5-(3-chloro-benzyl)-6-methyl-4-(2,4,6-trifluoro-phenyl)-pyridazine;
3-chloro-6-methyl-5-(3-methyl-benzyl)-4-(2,4,6-trifluoro-phenyl)-pyridazine;
3-chloro-5-(4-chloro-benzyl)-6-methyl-4-(2,4,6-trifluoro-phenyl)-pyridazine;
3-chloro-6-methyl-5-(4-methyl-benzyl)-4-(2,4,6-trifluoro-phenyl)-pyridazine;
3-chloro-5-(4-chloro-benzyl)-4-(2-chloro-6-fluoro-phenyl)-6-methyl-pyridazine;
3-chloro-4-(2-chloro-6-fluoro-phenyl)-6-methyl-5-(4-methyl-benzyl)-pyridazine;
3-chloro-5-(4-chloro-benzyl)-4-(2,6-difluoro-4-methoxy-phenyl)-6-methyl-pyridazine;
3-chloro-4-(2,6-difluoro-4-methoxy-phenyl)-6-methyl-5-(4-methyl-benzyl)-pyridazine;
3-chloro-5-(4-chloro-benzyl)-4-(3,5-dichloro-pyridin-2-yl)-6-methyl-pyridazine; and
3-chloro-4-(3,5-dichloro-pyridin-2-yl)-6-methyl-5-(4-methyl-benzyl)-pyridazine.

14. A process for the preparation of a compound of formula I.2, 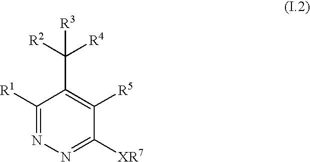
wherein R¹, R², R³, R⁴ and R⁵ are as defined for formula I, X is oxygen or sulfur and R⁷ is C₁-C₆alkyl or C₁-C₆haloalkyl, which comprises reacting a compound of formula I.1, 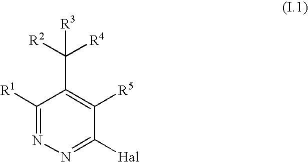
wherein R¹, R², R³, R⁴ and R⁵ are as defined for formula I and Hal is halogen with an alcohol or a thiol R⁷XH, wherein R⁷ is C₁-C₆alkyl or C₁-C₆haloalkyl and X is oxygen or sulfur, and a base or with a sodium alkoxide or thioalkoxide NaXR⁷, wherein X is oxygen or sulfur and R⁷ is C₁-C₆alkyl or C₁-C₆haloalkyl.

15. A process for the preparation of a compound of formula I.1, 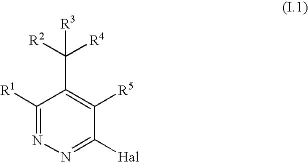
wherein R¹, R², R³, R⁴ and R⁵ are as defined for formula I and Hal is halogen, which comprises reacting a compound of formula I.3, 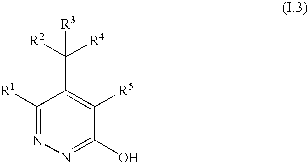
wherein R¹, R², R³, R⁴ and R⁵ are as defined for formula I, with a phosphorus oxyhalide PO(Hal)₃ or a thionyl halide SO(Hal)₂.

16. A process for the preparation of a compound of formula I.3, 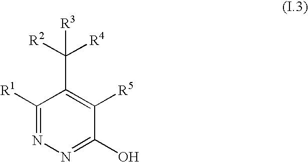
wherein R¹, R², R³, R⁴ and R⁵ are as defined for formula I, which comprises reacting a compound of formula II, 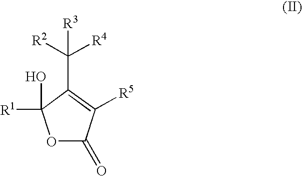
wherein R¹, R², R³, R⁴ and R⁵ are as defined for formula I, with a hydrazine derivative.

17. A process for the preparation of a compound of formula II, 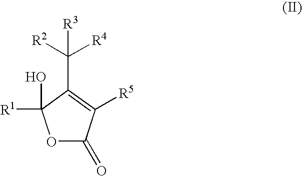
wherein R¹, R², R³, R⁴ and R⁵ are as defined for formula I, which comprises reacting a compound of formula III, 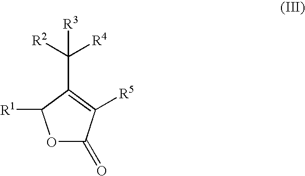
wherein R¹, R², R³, R⁴ and R⁵ are as defined for formula I, with oxygen, air or 3-chloroperbenzoic acid.

18. A process for the preparation of a compound of formula III, 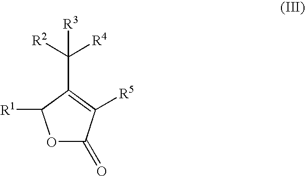
wherein R¹, R², R³, R⁴ and R⁵ are as defined for formula I, which comprises reacting a compound of formula IV, 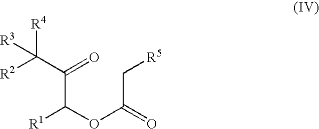
wherein R¹, R², R³, R⁴ and R⁵ are as defined for formula I, with a base.

19. A fungicidal composition for controlling or protecting against phytopathogenic microorganisms, comprising as active ingredient at least one compound as defined in claim 1, in free form or in agrochemically usable salt form, and at least one adjuvant.

20. The composition according to claim 19, which comprises at least one additional fungicidally active compound, preferably selected from the group consisting of azoles, pyrimidinyl carbinoles, 2-amino-pyrimidines, morpholines, anilinopyrimidines, pyrroles, phenylamides, benzimidazoles, dicarboximides, carboxamides, strobilurines, dithiocarbamates, N-halomethylthiotetrahydrophthalimides, copper-compounds, nitrophenols, organo-phosphorus-derivatives, pyridazines, triazolopyrimidines, carboxamides or benzamides.

21. The use of a compound as defined in claim 1 for controlling or preventing infestation of plants, harvested food crops, seeds or non-living materials by phytopathogenic microorganisms.

22. A method of controlling or preventing an infestation of crop plants, harvested food crops or non-living materials by phytopathogenic or spoilage microorganisms or organisms potentially harmful to man, which comprises the application of a compound as defined in claim 1, as active ingredient to the plant, to parts of the plants or to the locus thereof, to seeds or to any part of the non-living materials.

23. The method according to claim 22, wherein the phytopathogenic microorganisms are fungal organisms.

24. A composition comprising at least one compound as defined in claim 1 and/or at least one pharmaceutically acceptable salt thereof, at least one pharmaceutically acceptable carrier and/or at least one pharmaceutically acceptable diluent.

25. A compound as defined in claim 1 or a pharmaceutically acceptable salt thereof for use as a medicament.

26. A compound as defined in claim 1 or a pharmaceutically acceptable salt thereof for the treatment of cancer.

27. (canceled)

28. A method of treating cancer in a subject in need thereof, comprising administering a compound as defined in claim 1 to said subject in an amount effective to treat said cancer.
